(19) United States
(12) Patent Application Publication
(10) Pub. No.: US 2026/0051395 A1
(43) Pub. Date: Feb. 19, 2026

(54) SMART SCREEN APPLIANCE FOR PATIENT CARE

(71) Applicant: Mark Bluhm, Lake Elmo, MN (US)

(72) Inventor: Mark Bluhm, Lake Elmo, MN (US)

(21) Appl. No.: 19/299,658

(22) Filed: Aug. 14, 2025

Related U.S. Application Data
(60) Provisional application No. 63/683,010, filed on Aug. 14, 2024.

(51) Int. Cl. G16H 40/20; G06F 3/0488; G06Q 10/1093; G16H 10/60; G16H 20/10; G16H 80/00

(52) U.S. Cl. CPC G16H 40/20; G06F 3/0488; G06Q 10/1093; G16H 10/60; G16H 20/10; G16H 80/00

(57) ABSTRACT

A patient care system can include a smart screen appliance including a touchscreen display and processing circuitry that can be configured to execute a patient care application. A cloud-based infrastructure including a plurality of microservices deployed in a multi-tenant architecture, the plurality of microservices configured to manage patient care data. The cloud-based infrastructure can include a web tier. The smart screen appliance can be configured to communicate with the cloud-based infrastructure via the web tier. The cloud-based infrastructure can also include a plurality of customer-specific clusters. Each customer-specific cluster of the plurality of customer-specific clusters can include a control plane and a plurality of namespaces. Each namespace of the plurality of namespaces can include at least one microservice of the plurality of microservices and an associated database. An administrative console can be in communication with the cloud-based infrastructure through the web tier to access data from the plurality of microservices.

### PRIORITY

This application claims the benefit of priority to U.S. Provisional Patent Application Ser. No. 63/683,010, filed Aug. 14, 2024, which is incorporated by reference herein in its entirety.

### TECHNICAL FIELD

Examples described herein generally relate to a smart screen appliance or, more specifically, to a smart screen appliance for patient care.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 is a schematic diagram including an example system for a smart screen appliance for patient care.

FIG. 2 is a flowchart showing an example method for a smart screen appliance for patient care.

FIG. 3 is a schematic diagram including an example system for a smart screen appliance for patient care.

FIG. 4 is a schematic diagram including an example system for a smart screen appliance for patient care.

FIG. 5 is a schematic diagram including an example system for a smart screen appliance for patient care.

FIG. 6 is a schematic diagram including an example system for a smart screen appliance for patient care.

FIG. 7 is a schematic diagram including an example system for a smart screen appliance for patient care.

FIG. 8 is a schematic diagram including an example system for a smart screen appliance for patient care.

FIG. 9 is a schematic diagram including an example system for a smart screen appliance for patient care.

FIG. 10-FIG. 30 are graphical representations of example graphical user interfaces for a smart screen appliance for patient care.

FIG. 31 is a block diagram illustrating an example of a machine upon which one or more examples can be implemented.

FIG. 32 illustrates a flowchart of an example method relating to a patient care system.

### DETAILED DESCRIPTION

Nursing homes or assisted living centers are becoming more common as the population ages. Such care facilities present many unique challenges. As patients age, their ability to use personal technology (e.g., cell phones, computers, tablets, or the like) can become more challenging. Patient schedules can also be challenging for the patient or the staff to accommodate because there are so many conflicting schedules within the care facilities. Care-specific patients (e.g., memory care) can also have additional issues or problems, such as forgetting the names or faces of family members or care team members, forgetting important personal information, or forgetting the date or time of the present date.

To help address at least the problems mentioned above, the present disclosure can include a touchscreen application for a user who desires a simplified interface. The touchscreen application can be designed with a user-friendly interface featuring large, clear icons or text or a simple navigation system. This design can be particularly beneficial for users with cognitive challenges, as this interface makes the application easy to understand or use. The touchscreen application can permit users to see their daily schedule, tasks, time, weather, or other pertinent information. The application can also allow the user (e.g., the patient or care team members) to make voice or video calls or request help. The touchscreen application can allow emergency personnel to access medical information in emergencies. The touchscreen application platform can allow administrative personnel to schedule calendar events, tasks, messages, or the like. The touchscreen application can include different levels of administrative access to provide different capabilities for various persons interacting with the application. The touchscreen application aims to create a simple, large screen to help people with varying levels of cognitive challenges understand their day, their tasks, or their contacts or to help families or caregivers of those people who may be remote understand the medical care plan or schedule for the patient.

The touchscreen application can also be used in many situations where there is a desire to have a simple interface for end users where administrators can schedule or coordinate activities across single or multiple (groups) of users. Simple examples of this could be families with children in different locations (e.g., schools in different states), where the parent desires to be able to schedule or communicate across a group of people (e.g., children). In examples, the administrators can schedule events by floors, particular faith-based events, or the like. Another example would be a cruise ship where the cruise line could schedule activities for each passenger or allow them to communicate or provide information across groups of people. Similarly, the touchscreen application can be used for to ur groups, educational settings, communities (e.g., apartments, hotels, condominiums, senior living, assisted living, or special care facilities), health clubs, exercise clubs, or the like. Because the platform can be configured to provide medical or sensitive Pll information, the present disclosure is designed with robust security systems, including encryption or user authentication, to ensure compliance with the Health Insurance Portability or Accountability Act (HIPAA) or General Data Protection Regulation (GDPR) standards.

In other words, the touchscreen application can provide a safe, curated experience whereby the friends or family of vulnerable people or practitioners who care for them can collaborate on scheduling, task management, promoting social activities, or accessing critical information. The touchscreen application can provide an all-in-one platform that not only improves experiences for individuals with dementia but also reduces stress for their loved ones regarding practical concerns while equipping staff to provide even higher-quality care for residents.

The touchscreen application can be used in the home of a patient with dementia to help address some of the challenges experienced as a result of their condition. The touchscreen application can display basic information such as the date, time, weather, etc., including a simple list of tasks that they can check off through the day, a calendar of upcoming events with photos of relevant staff caregivers to avoid confusion, or the ability to initiate audio or video calls from a simple contact list. The touchscreen application can be configured so patients in care homes can use the smart screen to sign up for activities or events, select meals, view a noticeboard, or request help.

The touchscreen application can ensure that a person with dementia does not need to remember anything from any previous look at it. The touchscreen application can be configured to return to a default view after a period of inactivity. The touchscreen application can be configured to present clear, simple options for a person with dementia. In an emergency, a care home employee can scan a barcode on the screen with a mobile app (e.g., the VestaConnect mobile app) or touch the barcode, which can put the smart screen into emergency mode. In emergency mode, the touchscreen application can be configured to display the medical history of the patient, medication information, or other critical details, saving time during emergencies when every second matters. The medical information screen can also pull up documents related to a medical history of patients. For example, the documents can include pertinent medical information, such as, health care directives, do not resuscitate requests, or the like for each respective patient.

The touchscreen application can be configured to provide administrators of the care facility access to an administrative portal. The administrative portal can allow the administrators to manage residents individually or as groups. The administrators can set tasks, manage a digital noticeboard, arrange appointments, push voting options to residents to select meals, sign up for events, or more. Staff members can utilize the mobile app to scroll through to find a resident or access their information or scan barcodes on the smart screens. Family or friends can also communicate with the smart screen of the present disclosure via a mobile application (e.g., the VestaConnect app) to schedule visits, help manage appointments, stay in touch, or keep up the schedule of the patient. Setting up the touchscreen application in a care facility is a straightforward process that can include installing the application on the smart screens, configuring the administrative portal, or training staff or residents. The touchscreen application is designed to be intuitive or easy to use, reducing the need for extensive training.

The above discussion is intended to provide an overview of the subject matter of the present patent application. It is not intended to provide an exclusive or exhaustive explanation of the invention. The description below provides further information about the present patent application.

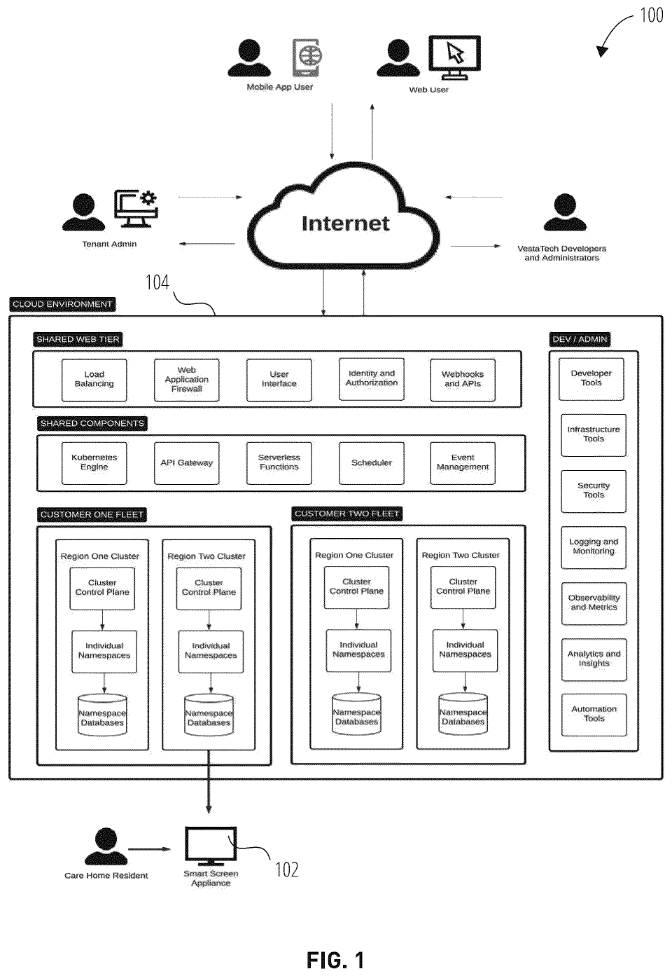

FIG. 1 is a schematic diagram including an example of a patient care system 100. The patient care system 100 can be configured to decrease frustrations or pain points related to patient care. The patient care system 100 can include a smart screen appliance 102. The smart screen appliance 102 can be a locked-down tablet (e.g., on the Apple or Android platform), monitor, computer, or other display. The smart screen appliance 102 can be configured to run in kiosk mode, which displays the touchscreen application (e.g., VestaConnect smart screen application) for users. The smart screen appliance 102 can include an endpoint lockdown agent that forces the device into kiosk mode, ensuring it only runs the application. The smart screen appliance 102 can feature a local cache, allowing certain services to operate offline during internet outages, such as displaying basic information, noticeboards, tasks, or calendar information. A queuing system can be included in the smart screen appliance 102 to enable synchronization once service is restored. When unassigned, the smart screen appliance 102 can prompt a user to set up an internet connection display, a barcode, or a unique alpha-numeric identifier for the assignment. Consumer admins can scan the barcode to add the smart screen appliance 102 to their account, while institutional admins can enter the alpha-numeric code to assign it to a primary user. In institutional settings, admins can disassociate a smart screen appliance 102 from a primary user, wiping all cached data or returning it to an unassigned state. The smart screen appliance 102 (or the patient care system 100) can include an endpoint agent that can be configured to disable all USB or other ports (e.g., all connection points available on the tablet that are not required to run the patient care system 100). The smart screen appliance 102 can include a feature allowing the primary user to touch an icon, revealing two large buttons for raising or lowering the volume, specifically for the chat service.

The patient care system 100 can include an application 104 (e.g., the VestaConnect Application) that can be configured to run on the smart screen appliance 102. The primary user can use the application 104. The application 104 can be paired with an endpoint lockdown agent to force the smart screen appliance 102 into kiosk mode or lock the smart screen appliance 102 down such that it only runs the application 104.

In examples, the patient care system 100 can include various user types at the top, including Mobile App User, Web User, Tenant Admin, or Developers or Administrators. These users can interact with the system through the Internet, represented by a cloud icon.

The patient care system 100 can include a cloud environment with multiple layers or components. The Shared Web Tier can include Load Balancing, a Web Application Firewall, a User Interface, Identity or Authorization, or Webhooks or APIs. These components can handle incoming requests or user authentication.

The Shared Components layer can contain Kubernetes Engine, API Gateway, Serverless Functions, Scheduler, or Event Management. These elements can provide the core infrastructure for the system's operation.

The patient care system 100 can include two customer fleets: Customer One Fleet or Customer Two Fleet. Each fleet can contain multiple regional clusters. For example, Customer One Fleet can include Region One Cluster or Region Two Cluster. Each cluster can consist of a Cluster Control Plane, Individual Namespaces, or Namespace Databases. This structure can allow for isolated or secure data management for each customer.

The patient care system 100 can also include a developer or admin section that can include various system management or development tools. These tools can include Developer Tools, Infrastructure Tools, Security Tools, Logging or Monitoring, Observability or Metrics, Analytics or Insights, or Automation Tools.

The architecture of the patient care system 100 can be configured to support multi-tenancy, with each customer having an isolated environment within the shared infrastructure. Regional clusters or namespaces can enable data segregation or customization for different care home facilities or organizations.

This system architecture can allow for scalability, security, or flexibility in managing care home operations across multiple locations or for various users, from residents to administrators.

The Consumer Admin Console can be an application that a consumer can leverage to create an account for a primary user, onboard a smart screen, sign up for a subscription service, or manage the ongoing experience for the primary user. There can be three options for the computer admin counsole: a web application, an iOS mobile application, or an Android mobile application. This can be the same application used by connected user admins or connected users connecting with a primary user in a care home but with all privileges removed by default until granted by an institutional admin. A feature of this application can be the ability to toggle between multiple primary users or to have the appropriate permissions for each.

The Institutional Admin Console can be an application leveraged by institutional admins to onboard screens or primary users, create groups of screens or users, manage services on behalf of individuals or groups of users, invite people to connect with primary users, etc. In examples, a web application can be used for the institutional admin console.

The Institutional Staff Application can take the form of Android or IOS mobile applications. This mobile app can contain all the information on the primary users in a given institution. Staff can be granted access to all or a subset of users based on permissions set by institutional admins. This application can allow staff members to manually search for individual primary users or go to a user's screen or scan a barcode, which can then bring up a primary user's account. The staff member can use this application to view or edit a primary user's calendar, set or manage tasks, or access medical information, provided that the institutional admin grants these permissions. The application can have a feature whereby a staff member can press a button once a primary user's account is up, which can display all relevant medical information on the primary user's screen. Alternatively, a staff member can go to a screen, touch the barcode, or be prompted to enter a PIN that can bring up the primary user's medical information on the screen. In addition to controlling basic settings, staff can manage PINs within the application.

The application 104 admin console can be an appropriate administrative tool or tool set with which all of the above can be managed, and can include other components associated with running a highly available, secure, multi-tenant software-as-a-service platform.

The application 104 platform can incorporate several features or services to support residents or staff in care facilities. The Basic Information service can remind residents of their identity, location, date or time, or weather information. The Collaborative Activity Sequencing feature can allow scheduling or managing activities, including photo reminders of staff or third parties, with edit rights shared between staff or family members. This feature can generate notifications or produce calendar reports, with the ability to export data using industry-standard formats (e.g., CSV, PDF, TXT, or the like).

The Task Assignment and Voting service can enable the creation of recurring tasks or votes with customizable expiration settings or persistent confirmation options. The Bulletin Board feature can allow for sign-ups with persistent affirmation, automatic calendar addition, conflict notifications, or information-only reminders with expiration or recurrence capabilities.

Audio/Video Chat functionality can provide uni- or bi-directional initiation options, with the ability to warn if an activity is underway or a secondary screen for contacts. The platform can include a Request for Help feature or an Emergency Medical Info service, which can store patient timelines, medical issues, or medication information. It can also capture a photo when an emergency code is entered or send notifications for specific events. Connected users (people who have agreed to be called) can configure their availability times to be called. Also, the administrators can remove the ability for a resident to make calls completely.

The system supports two facility interfaces: resident screens or common room areas. Resident apps can be available for Apple or Android devices. Additional features can include analytics on management console usage, a help request button for residents, or user-type open-ended attributes for policy creation. The platform can incorporate a primary device database from ERP, mapping MAC addresses to device information or application versions.

A directory function for common groups or actions based on shared attributes across users or sites can be included. The system can also manage non-registered user data, potentially with email verification or time-bound consent processes. A Contact Management service can add, edit, or remove contacts with clear statements on data storage or usage.

The platform can use barcodes on screens tied to MAC addresses to display primary user data on institutional user devices based on pre-authorization. The platform can include logo display capabilities (e.g., images, diagrams, or symbols to help guide an end-user). The platform can also include functionality to move residents between facilities using a primary resident database at the cluster level, accessible only to cluster admins. The functionality to move residents between facilities can include an event-driven cloud function that migrates data or manages resources accordingly.

The platform can also include an image or picture service. Anyone connected to the user can upload images or pictures that can be shown on the display. The pictures can rotate automatically to shuffle all uploaded images. In examples, the images or pictures can be scanned through by a user by swiping their finger on the photo to go to the next picture, or the other direction to go back.

The platform can also include a “document” service. The document system can include monthly calendars for things like menus, religious services, activities, or the like. The documents can be loaded in the system and pulled up by just touching the document button, and a list of available documents shows up with a friendly name, e.g., Food Menu, Baseball Schedule, football schedule, dance claims, or the like. As patients sign up for various events, they can receive documents related to activities on their schedule via the document service. For example, the facility or family admin can load documents, and they can be scheduled in advance. The documents can also include tags, such that the document related to the pickleball league is sent to all people who have signed up for the pickleball league. Some documents can be hidden from the patient but available for access to family members, healthcare professionals, or the like. For example, families can load pertinent information about the resident, like background information, wills, care notes, or the like. This system can help keep all documents, whether private or public, in a single document management system for each patient.

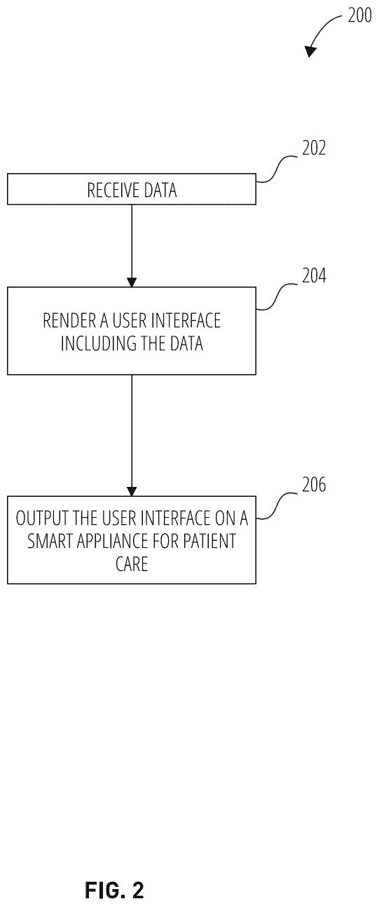

FIG. 2 is a flowchart showing an example method 200 for a smart screen appliance for patient care (e.g., smart screen appliance 102, see FIG. 1). Although the example method 200 depicts a particular sequence of operations, the sequence may be altered without departing from the scope of the present disclosure. For example, some of the operations depicted may be performed in parallel or in a different sequence that does not materially affect the function of the method 200. In other examples, different components of an example device or system that implements the method 200 may perform functions at substantially the same time or in a specific sequence. Moreover, the method 200 can include more operations or can omit any of operations 202-206.

According to some examples, the method includes receiving data at operation 202. For example, the data received in operation 202 can include information provided by the Basic Information service, such as reminders for residents of their identity, location, date or time, or weather information. The data can also include the Collaborative Activity Sequencing feature can allow scheduling or managing activities, including photo reminders of staff or third parties, with edit rights shared between staff or family members. The data can also include notifications or produce calendar reports, with the ability to export data to industry standard formats (e.g., csv, pdf, txt, or the like).

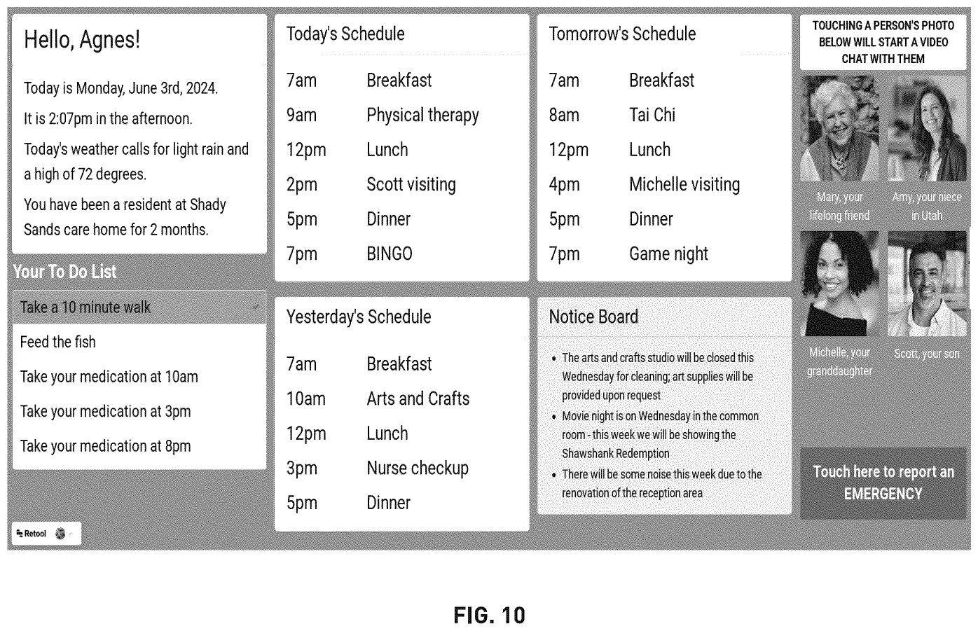

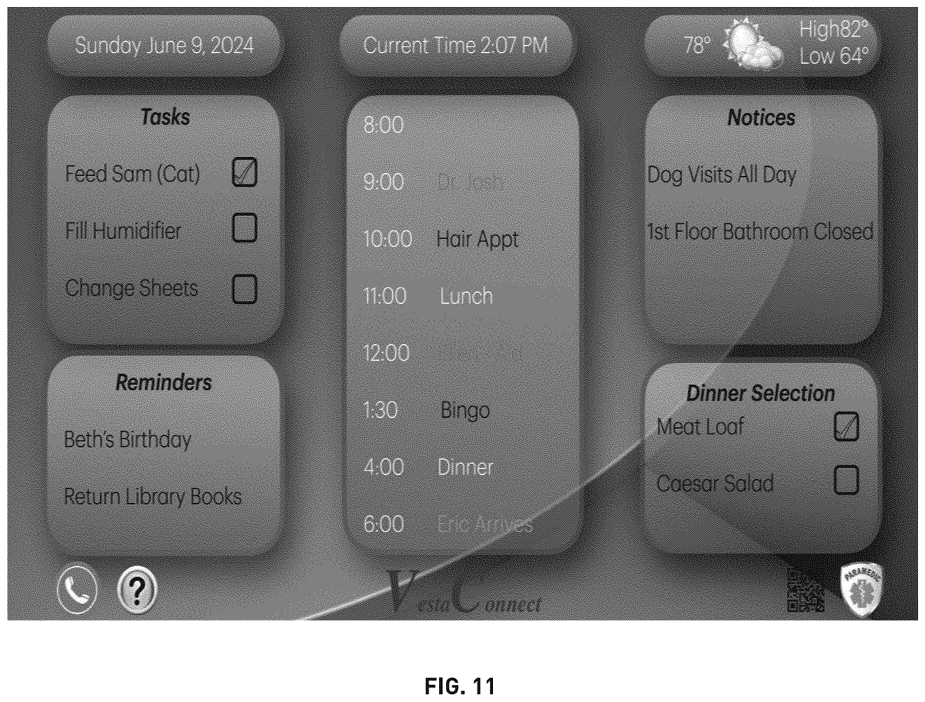

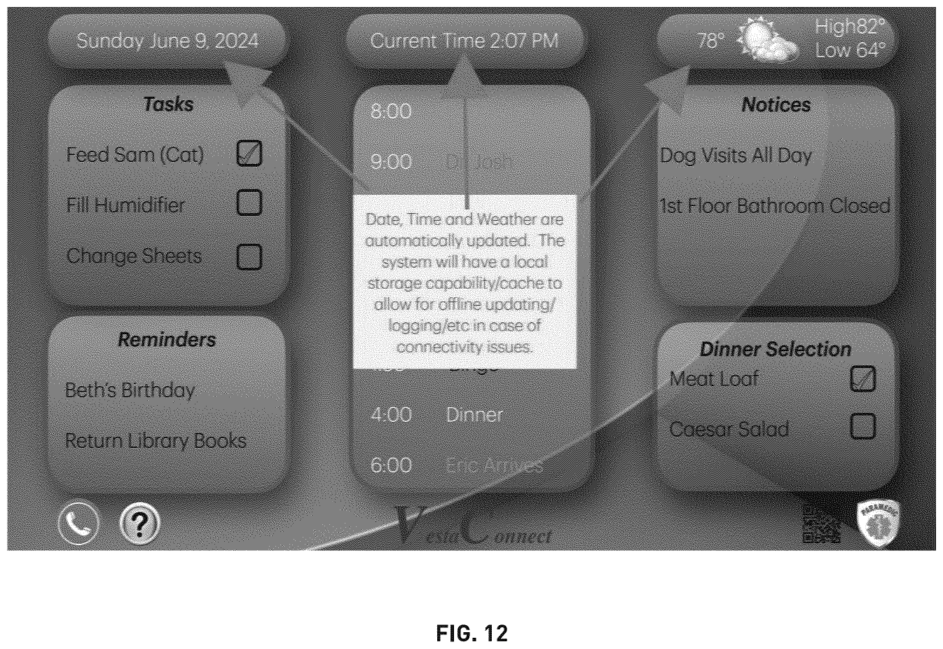

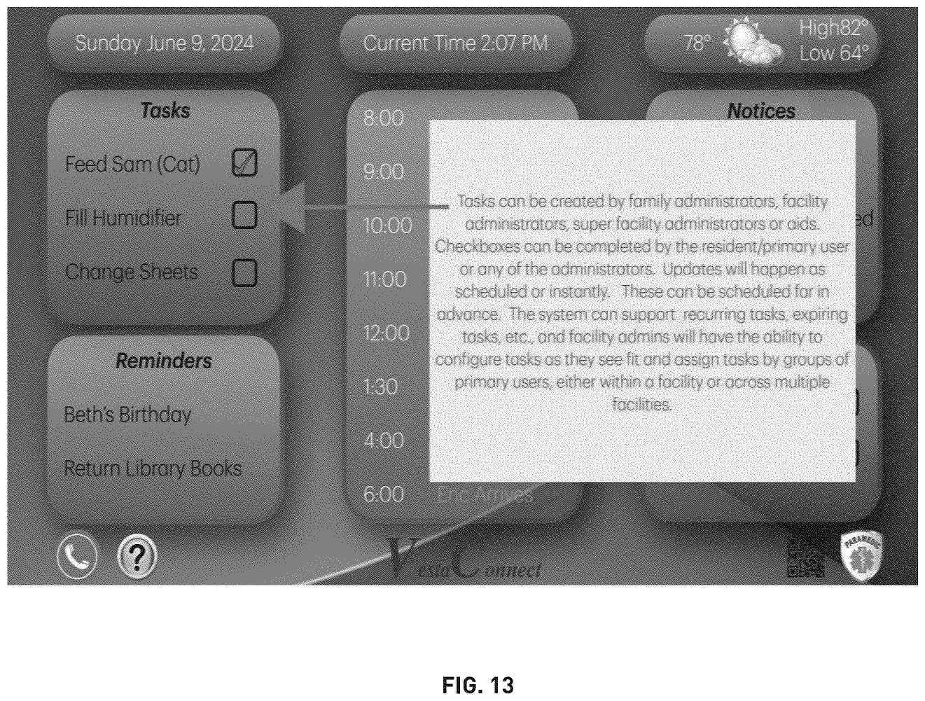

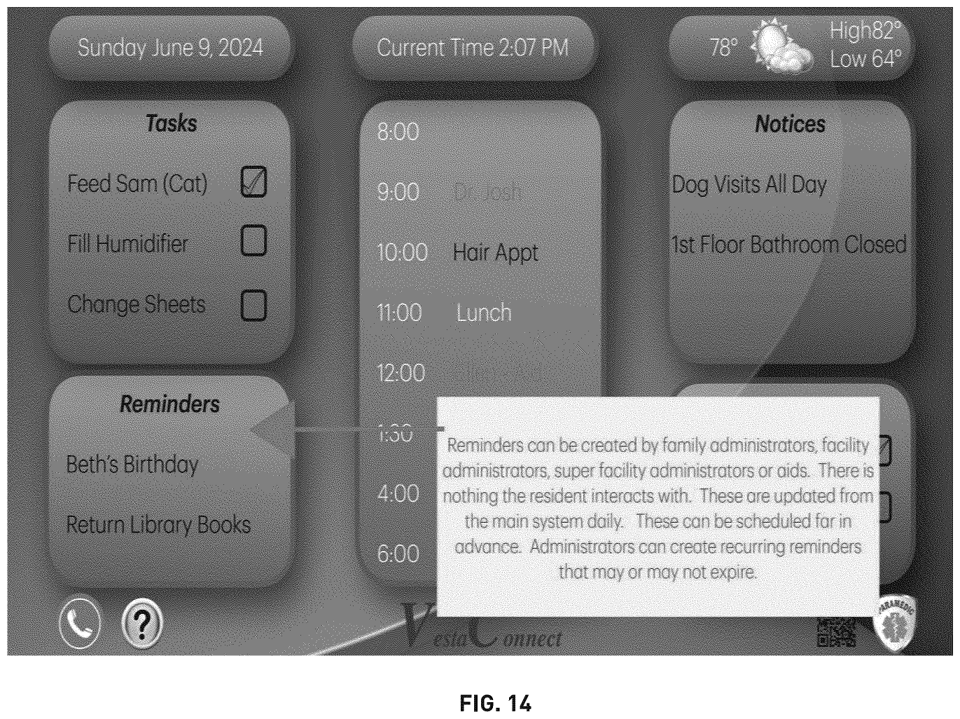

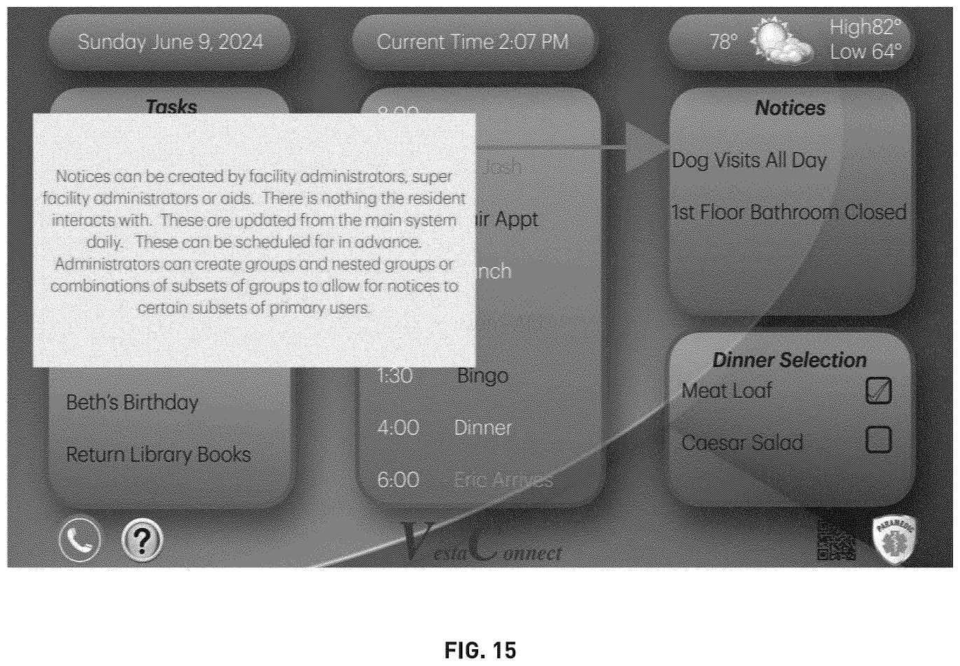

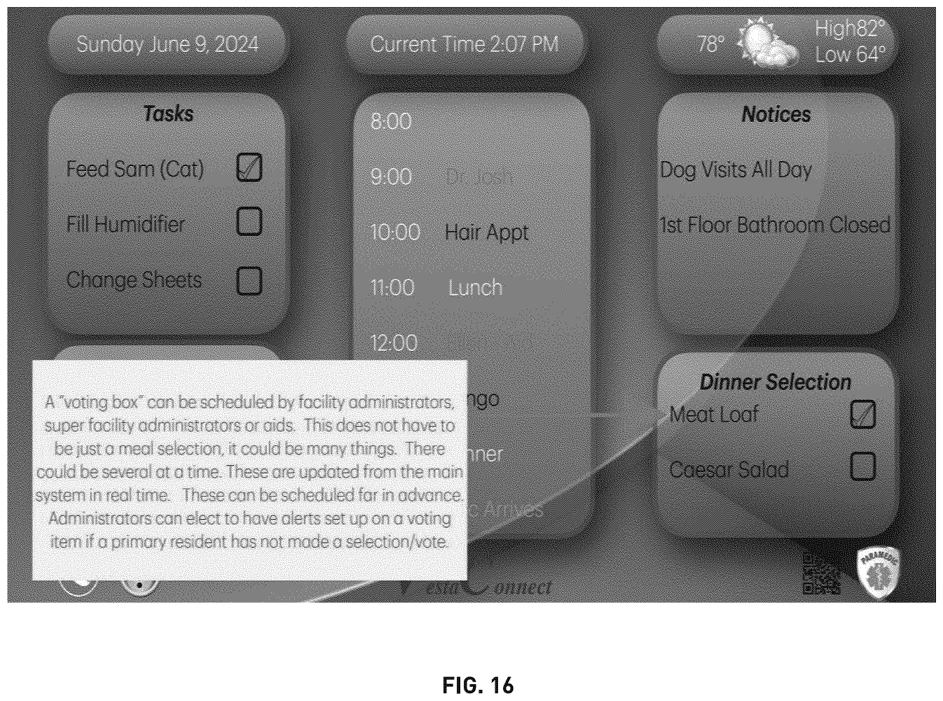

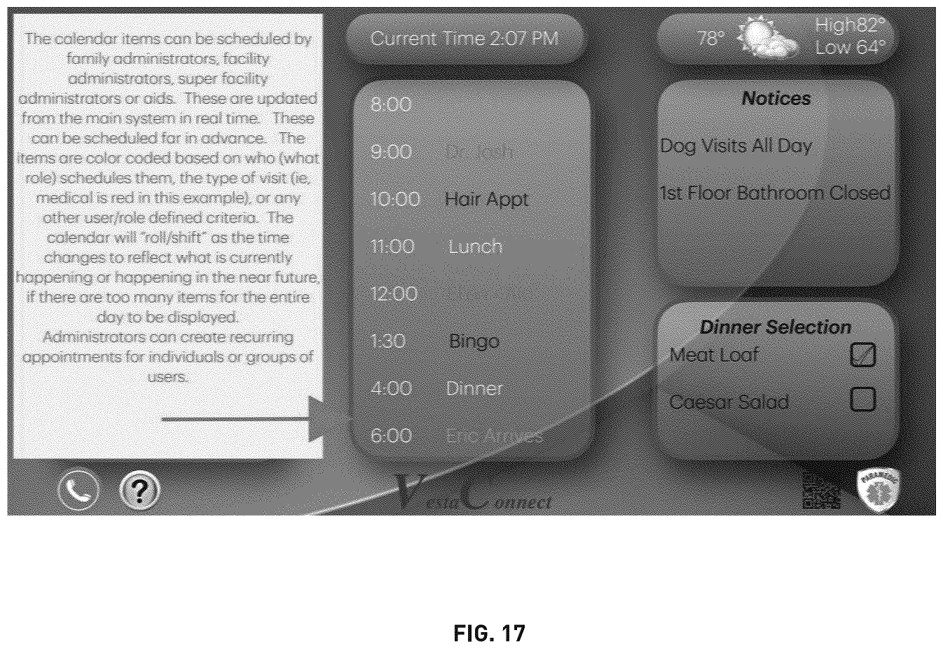

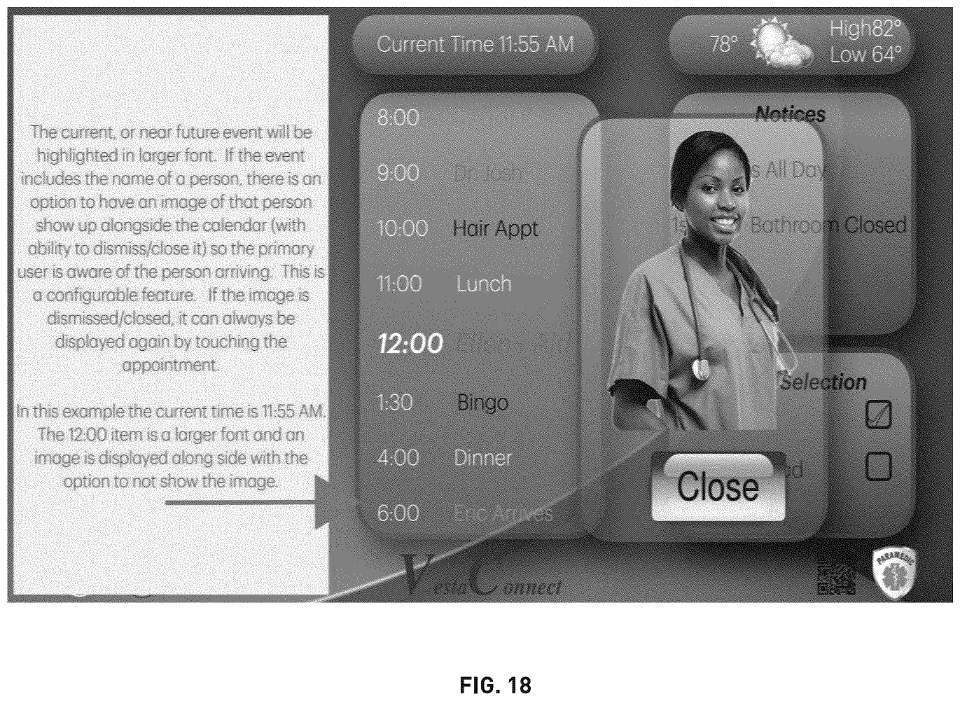

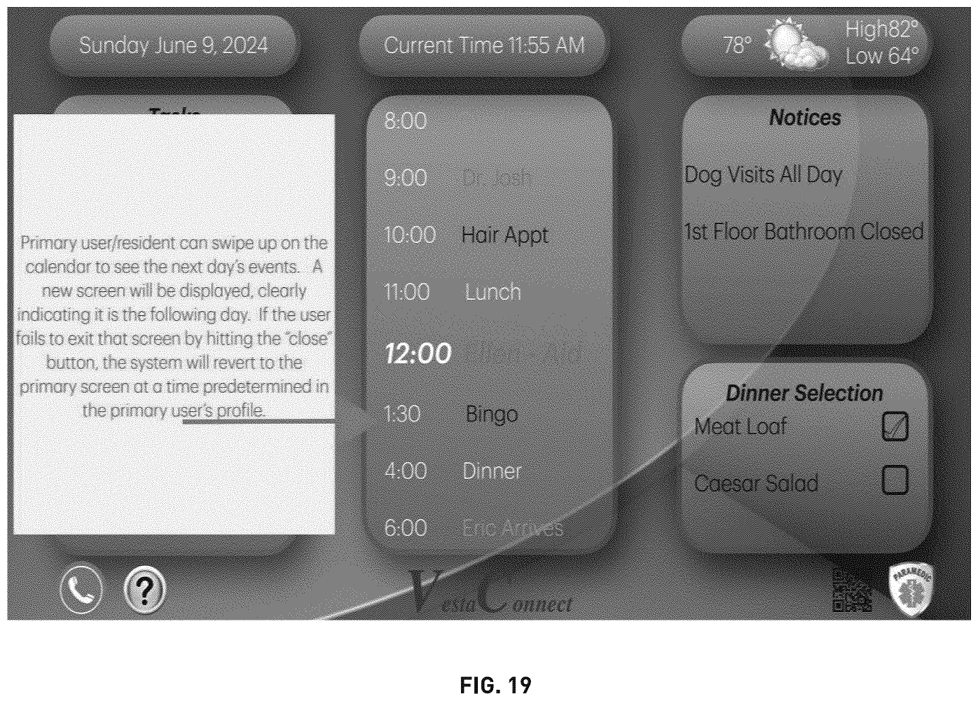

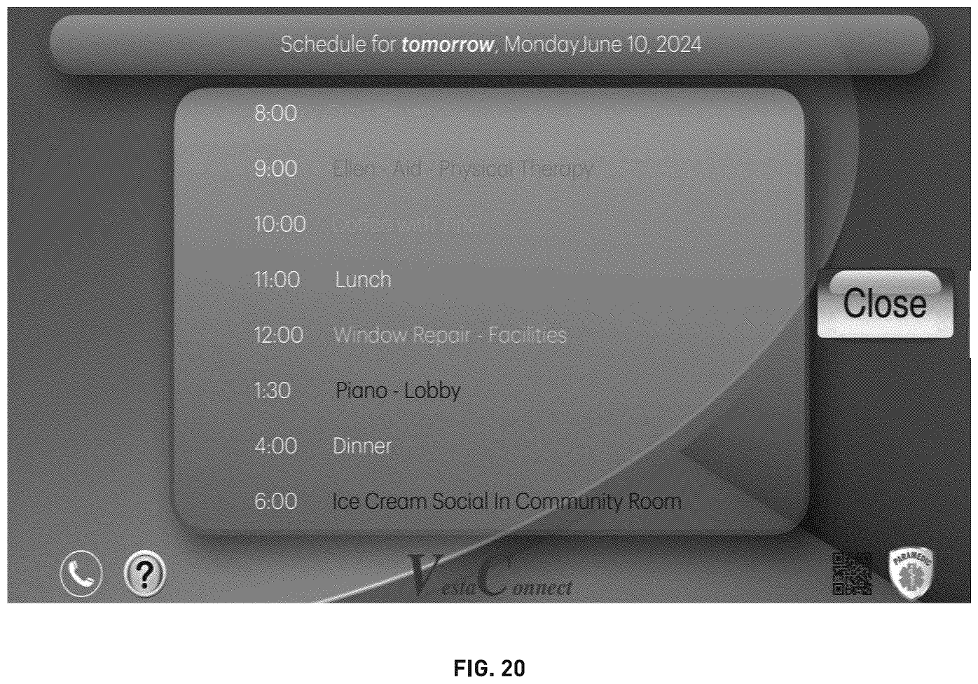

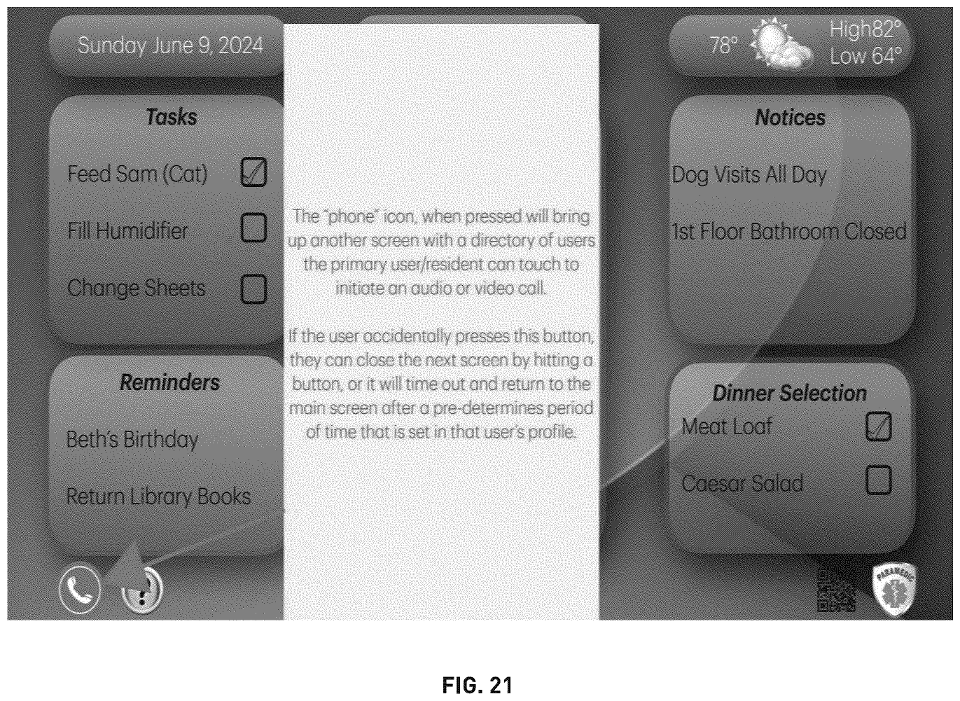

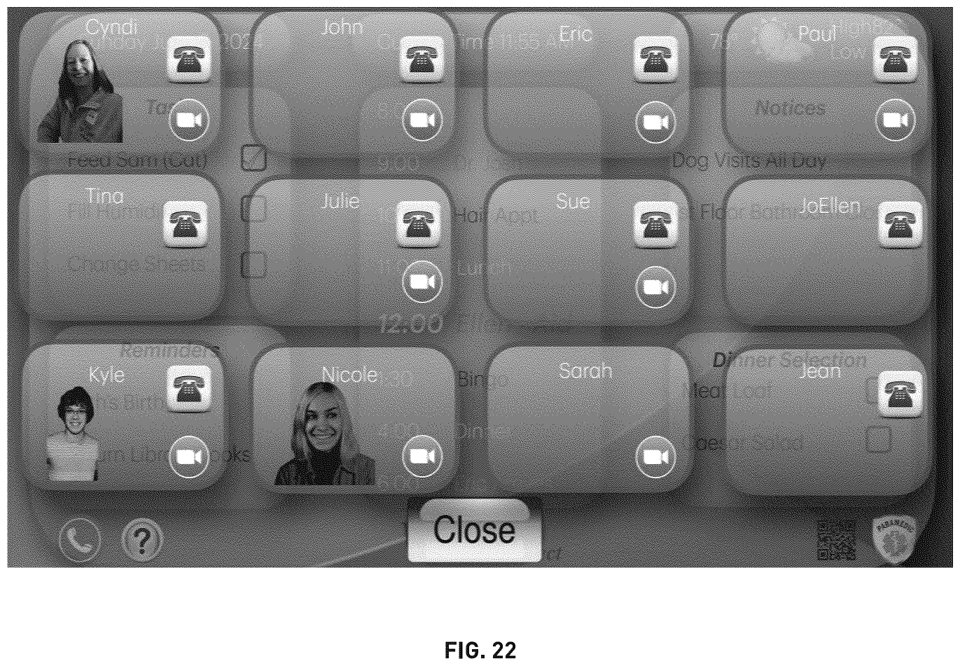

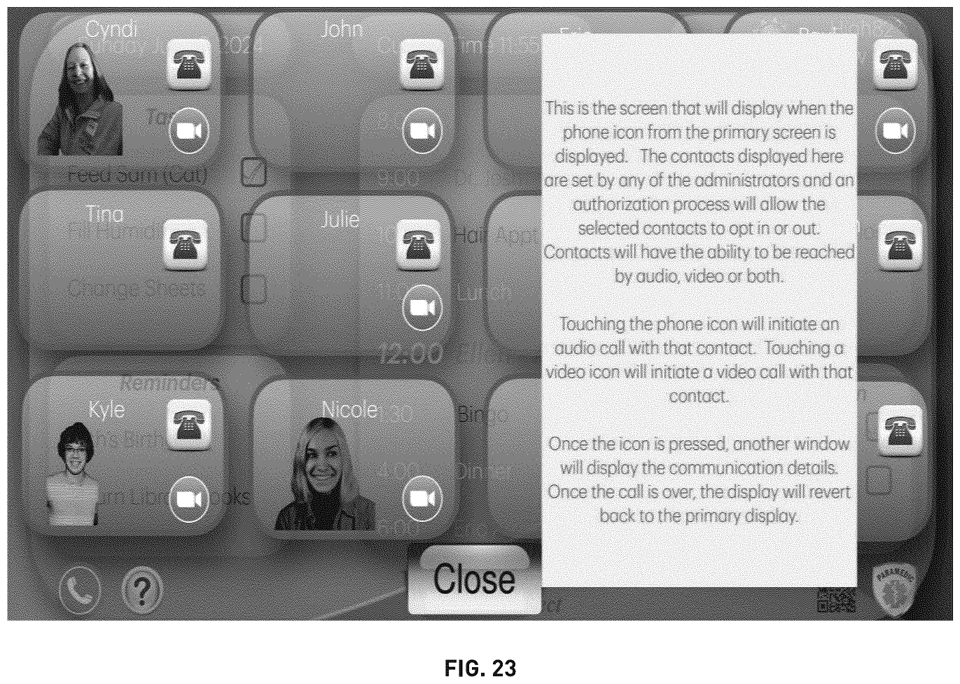

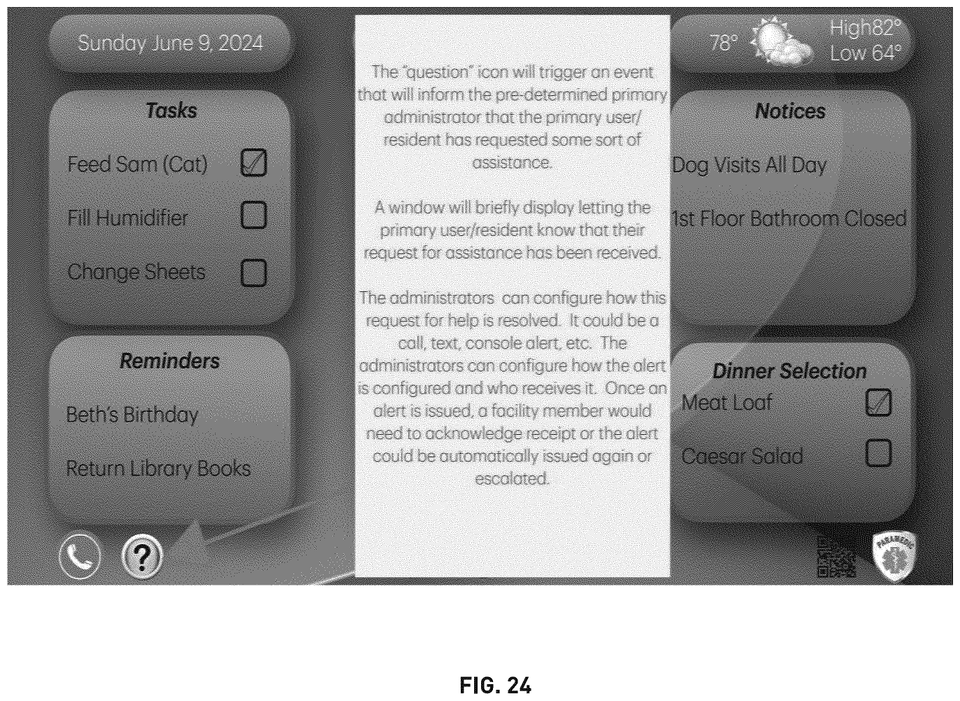

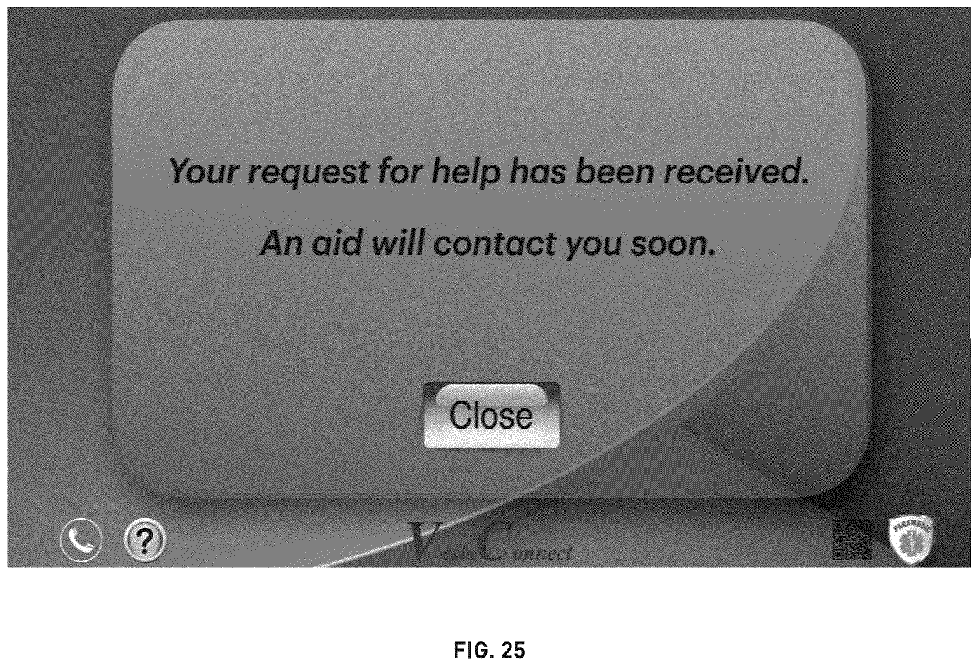

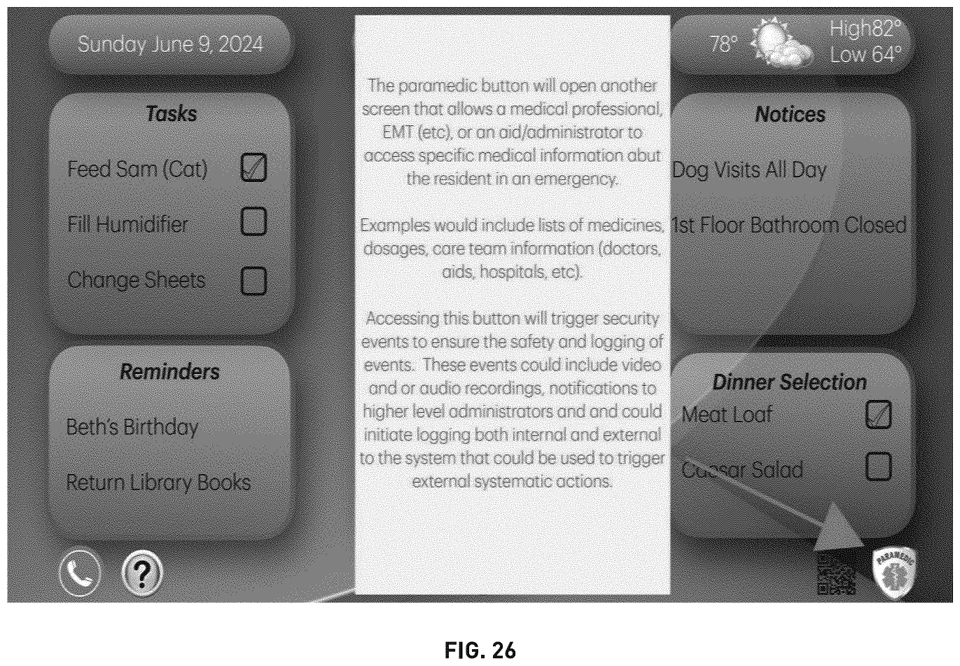

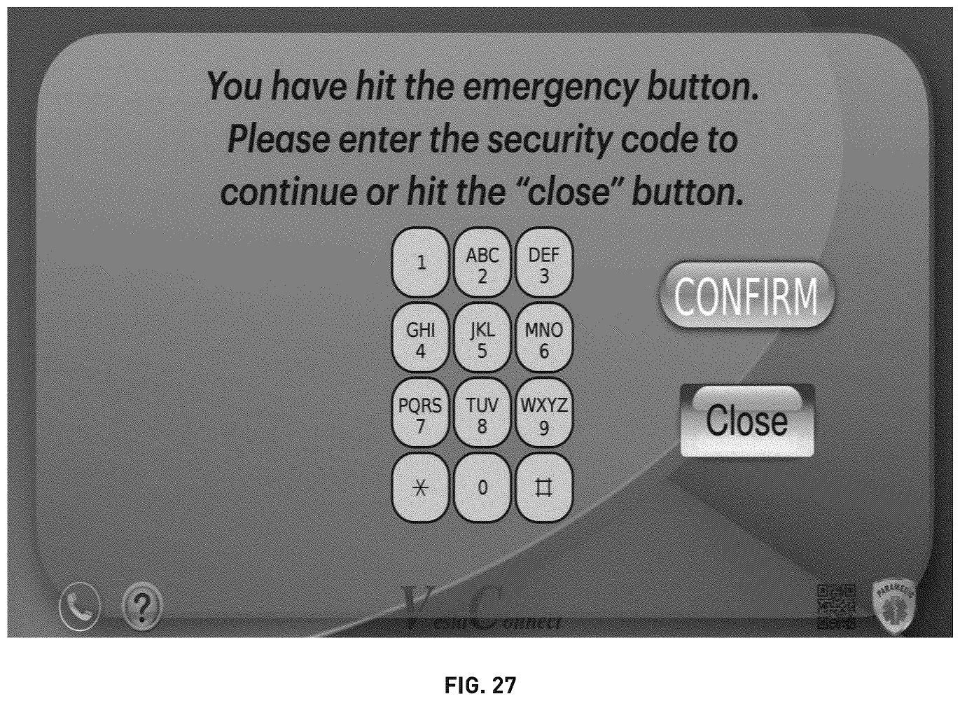

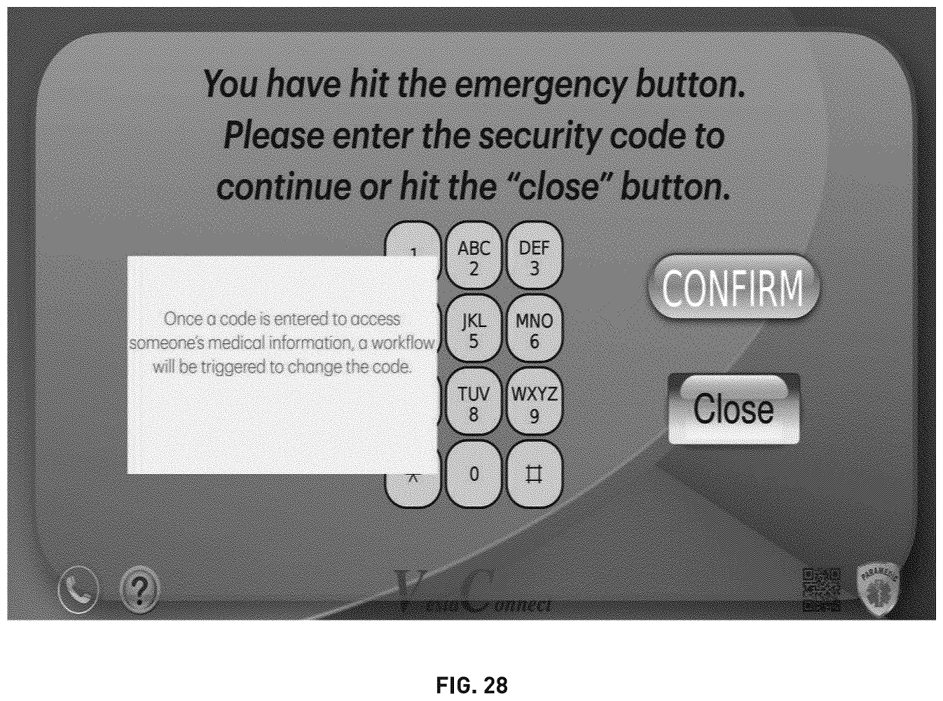

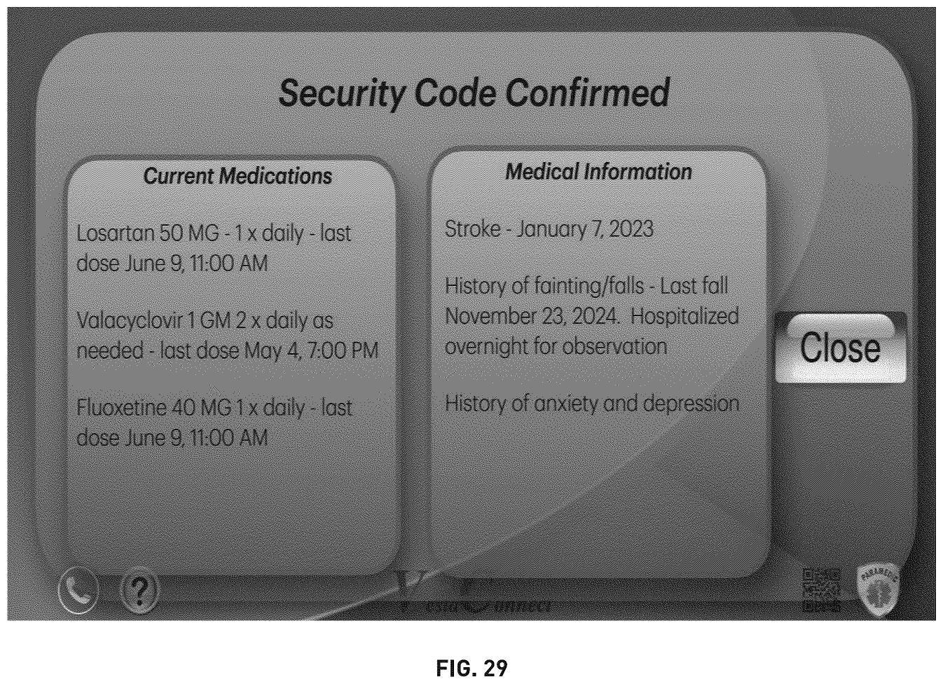

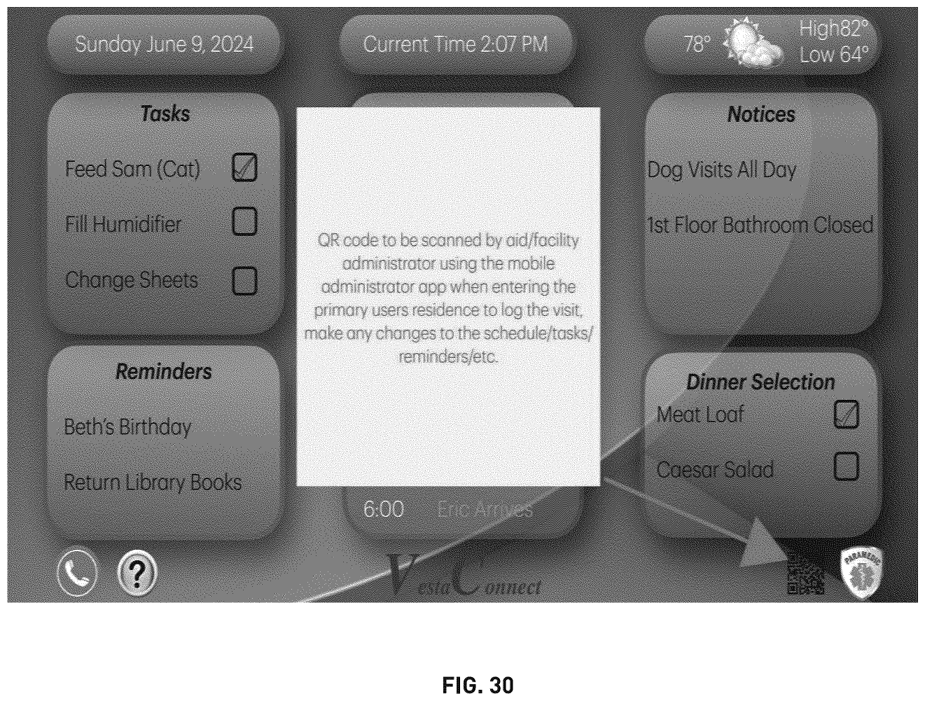

According to some examples, the method includes rendering a user interface including the data at operation 204. Examples of the user interface are shown in FIG. 10-FIG. 30.

According to some examples, the method includes outputting the user interface on a smart appliance (e.g., the smart screen appliance 102, see FIG. 1) for patient care at operation 206. As discussed herein, the smart screen appliance 102 can be engageable by users to alter the information displayed, contact set contacts, contact the medical team, communicate with other users of the medical team, or the like.

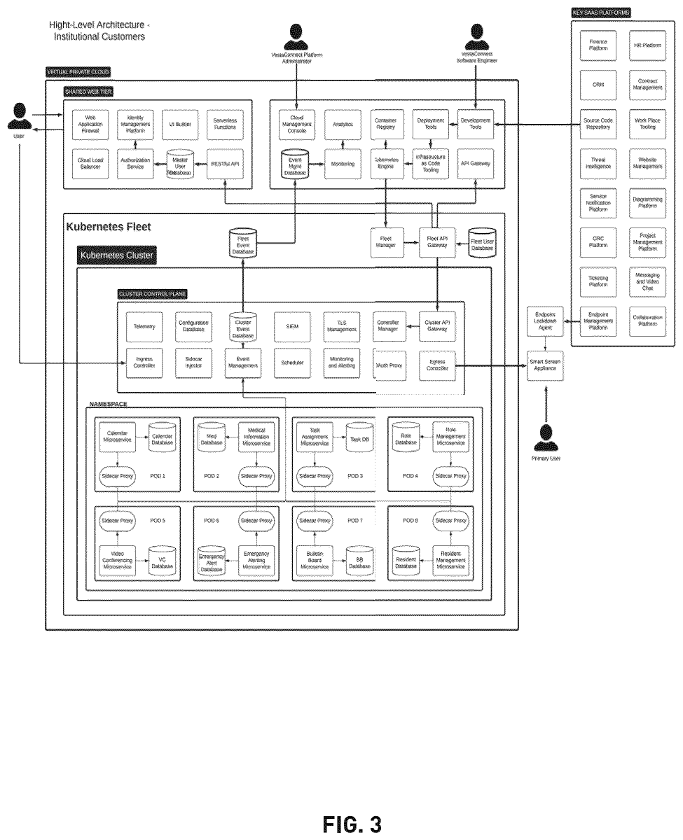

FIG. 3 is a system architecture diagram showing a high-level view of a cloud-based care home management system, according to some examples.

The system (e.g., patient care system 100, see FIG. 1) can include multiple user types interacting with it through the Internet. These user types can include Users, application Administrators, application software engineers, or the like. The Internet can serve as the central point of access for all users to connect to the virtual private cloud.

The virtual private cloud can contain several layers of components or services. The Shared Web Tier can include a Web Application Firewall for security, an Identity Management Platform for user authentication, a UI Builder for creating user interfaces, Serverless Functions for executing code without managing servers, a Cloud Load Balancer for distributing incoming network traffic, or Authorization Service for controlling access to the system. These components can handle incoming requests, provide security, manage user interactions, or facilitate communication with other system components.

The Shared Components layer can include Cloud Management Console, Analytics, Container Registry, Kubernetes Engine, or API Gateway. These elements can provide the core infrastructure for the system's operation, enabling containerized application deployment, API management, monitoring, or analytics.

The system can support a Kubernetes Fleet, which can include a Kubernetes Cluster. The Kubernetes Cluster can include a Cluster Control Plane or a Namespace. The Cluster Control Plane can include components such as Telemetry, Configuration Database, Ingress Controller, or Sidecar Injector to manage the overall operation of the cluster.

Each Namespace can contain multiple microservices or associated databases, such as Calendar Microservice, Medical Information Microservice, Task Assignment Microservice, or Role Management Microservice. Each microservice can be represented as a pod with a sidecar proxy, allowing for modular or scalable application architecture.

SaaS Platforms can include various tools for system management or integration. These tools can include a Finance Platform for managing financial data, an HR Platform for managing human resources, a CRM for managing customer relationships, a Source Code Repository for storing or managing source code, or other useful business tools. These components can support the development, deployment, or maintenance of the system.

A Primary User can interact with a Smart Screen Appliance. This appliance can connect to the Virtual Private Cloud through an Endpoint Lockdown Agent, potentially allowing the resident to access the system's services through a dedicated interface.

The architecture can support multi-tenancy, with each customer having an isolated environment within the shared infrastructure. Kubernetes clusters or namespaces can enable data segregation or customization for different care home facilities or organizations.

This system architecture can allow for scalability, security, or flexibility in managing care home operations across multiple locations or for various users, from residents to administrators. The layered approach can provide a separation of concerns, with shared components handling standard functionalities or customer-specific resources isolated in their respective namespaces.

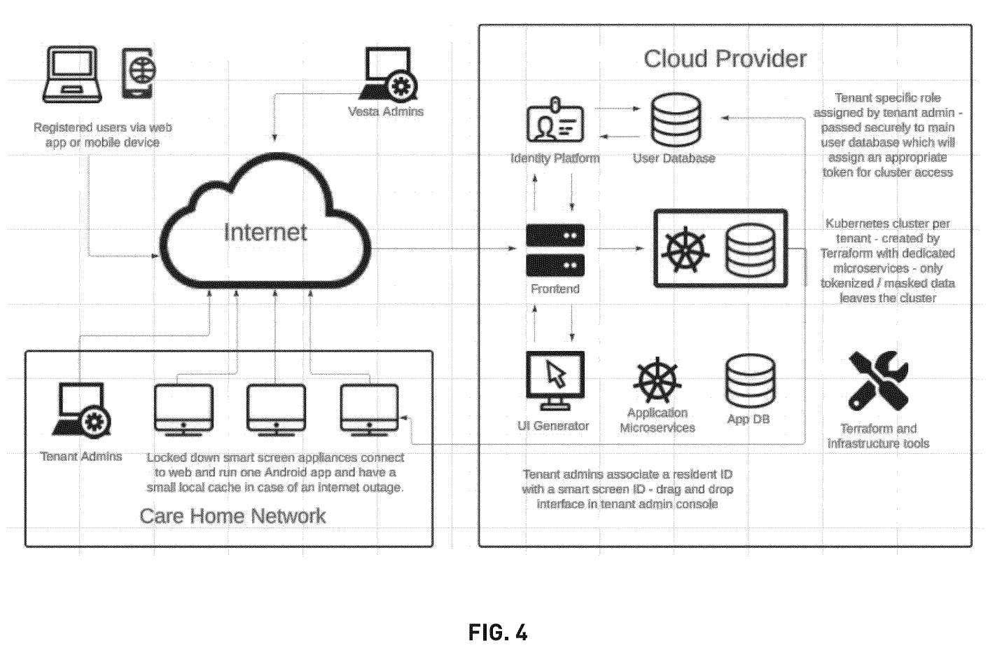

FIG. 4 is a system architecture diagram illustrating the components or connections of a cloud-based patient care home management system, according to some examples.

The system can be divided into three main sections: Cloud Provider, Care Home Network, and Internet. The Cloud Provider can include an Identity Platform that can interface with a User Database. The Identity Platform can connect to a Frontend component, which can connect to a UI Generator or Application Microservices. These microservices can interact with an App DB. The UI Generator or Application Microservices can be represented within a container symbol, suggesting they can be deployed in a containerized environment. The Care Home Network section can show multiple locked-down smart screen appliances. These devices can connect to the web or run a single Android app. They can also maintain a small local cache for use during internet outages. The Internet section can be an intermediary, connecting the Cloud Provider to the Care Home Network or other external components. It can also connect to Registered users via web apps, mobile devices, or Admins.

The Tenant Admins can associate a resident ID with a smart screen ID using a drag-and-drop interface in the tenant admin console. This association can assign a tenant-specific role, which can then be securely passed to the primary user database. The database can subsequently assign an appropriate token for cluster access.

The system can utilize Kubernetes clusters, with one cluster per tenant. An infrastructure such as code software can create these clusters, which can contain dedicated microservices. In examples, only tokenized or masked data can leave the cluster, suggesting a focus on data privacy or security.

Additional components shown can include tools for Transform or Infrastructure or icons representing Tenant-specific roles or Kubernetes clusters.

The architecture can support a multi-tenant environment with secure, isolated data processing per tenant while providing a unified management interface through the cloud provider.

The primary user can view calendar information, scroll into the future, or see color-coded events. The primary user can view the noticeboard, mark tasks as complete, or vote where voting buttons are available. The primary user can receive reminders or can consume information on current date, weather, or time. The primary user can request help or initiate video/voice calls by clicking icons. The primary user can access a subset of services during internet outages or view pop-up messages sent by admins/staff. The primary user's screen can remain consistently on, or the primary user can view a QR code for emergencies.

Consumer admins can manage connected users or contacts, create profiles for primary users, or manage calendar events or tasks. Consumer admins can serve as admins for multiple primary users, change smart screens, set limitations on calling times, or input medical history. Consumer admins can create or change PINs to access medical information, receive notifications about screen status or task completion, or set basic permissions for connected users.

Institutional admins can manage the lifecycle of primary users or screens, create or manage groups, configure default options, or onboard connected users. Institutional admins can manage calendars or tasks for individuals or groups, manage the noticeboard, or handle institutional staff accounts. Institutional admins can also control medical information access, contact management, or configuration of various system features, such as the help button or video/audio calling options.

Institutional staff can access primary user information through barcode scanning or manual search. The Institutional staff can manage calendars or tasks within set permissions, access medical information with proper authorization, or manage noticeboard items. When employed by multiple facilities, institutional staff can have standard account management capabilities or toggle between institutions or accounts.

Connected user admins can manage calendars or tasks for primary users within set permissions. The connected user admins can add connected users or contacts, access medical information, view the primary user's screen, or receive video/audio calls. Connected user admins can have basic account management capabilities or toggle between primary users.

Connected users can have viewing rights for calendars, tasks, noticeboards, or voting buttons. Connected users can receive audio/video calls from the primary user or perform basic account management. Contacts (e.g., easily contactable via the user interface of the smart screen appliance 102, see FIG. 1) can receive audio or video calls from the primary user or perform basic account management.

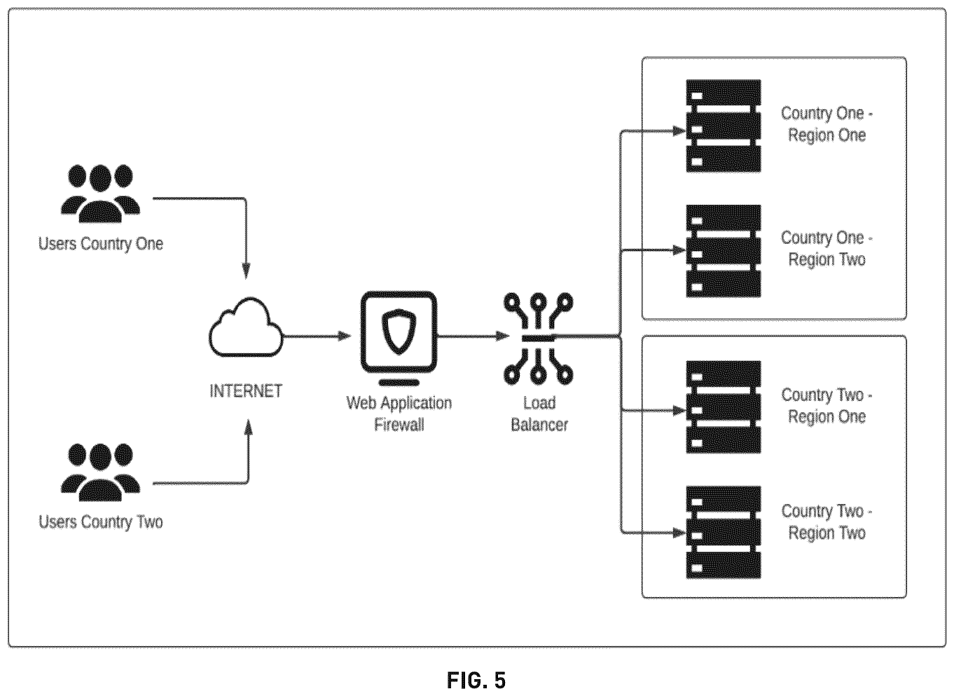

FIG. 5 is a schematic diagram including an example system for the patient care system 100. The cloud infrastructure that underpins the application platform runs in a virtual private network within a public cloud environment. The cloud infrastructure that underpins the application platform can run in a virtual private cloud within a public cloud environment. A web application firewall can mediate all access to the application or protect against distributed denial of service or bot attacks. Access management policies can also be in place to prevent brute force or other attacks.

The application 104 (FIG. 1) can have full redundancy across national regions or run in a live/live configuration. User access can be via a load balancer. The platform can maintain high service availability even if one of the in-country regions suffers a major sustained outage or other degradation of service.

Without multi-factor authentication, there can be no access to any component of the application 104 platform or any back office, internal IT, or enterprise systems, whether by employees, users, or any relevant third parties. Administrative access to underlying infrastructure can be via a privileged access management system with session recording. All activity can be logged, or logs can be sent to a tightly controlled centralized aggregation system to avoid any possibility of tampering. Logs can be actively monitored for suspicious activity or indications of compromise. Attempted access from new devices on new networks, where anomalous behavior is detected—unexpected geography, unusual time—can force MFA or revocation of relevant tokens.

Once the application is installed on a pre-certified touchscreen endpoint for use by a primary user, an endpoint management agent can place the device into kiosk mode or lock it down to only run the application. Due to strict safe listing policies, no other applications or executables can be initiated. Network protocols can be configured such that a smart screen can only connect to the application cloud once connected to the internet. The endpoint management agent can monitor the device, or endpoints can have a small local cache to ensure services are running locally in the event of an internet outage. Administrators can be notified if internet connectivity is lost, but a primary user can still display basic services. Asynchronicity resulting from an internet outage can be managed with a robust message management microservice to resync endpoints once connectivity is restored.

Upon installing or powering up an application smart screen for the first time, the installer can be promoted to connect to the internet. Once an internet connection is established, the smart screen can connect to the cloud infrastructure of the application, or the smart screen can display a QR code or instructions for manual registration. Once the QR code is scanned, the screen can be added to the appropriate application account. At that point, the screen can be visible in the admin console, where it can appear as an “unassigned screen” or then be linked to a primary user. There can be a manual process to add screens within the admin console. A smart screen can display information for a particular primary user for as long as it is linked to the user. Admins can remove a primary user from a screen, at which point the screen can clear its local cache or return to an “unassigned” status. It can then be linked to a new primary user.

The application can be designed to ensure the privacy of all user data, particularly primary users for whom the platform may contain personal health information or who, by circumstance, may be vulnerable or otherwise be in a vulnerable situation. The application can also have two distinct types of customers—consumer or institutional—or can work to ensure an appropriate level of segmentation of the data associated with each. Regardless of customer type, though, the platform can be architected so that no one at the application, regardless of their administrative privilege, can access any personally identifiable information—health-related or otherwise—about primary users. The application systems, including the central ID management service, can only contain ID numbers or non-personally identifiable or tokenized data about primary users.

The application can be a multi-tenant cloud service that operates as a virtual private network. Front-end components that do not hold any sensitive information can be shared. The backend of the system can be containerized via Kubernetes, as the application service can be comprised of several independent microservices that can be tailored for an individual primary user's needs based on the nuances of their particular situation. For segmentation for consumer customers, there can be dedicated redundant pairs of Kubernetes clusters per country running in a live/live configuration to ensure high availability. Each cluster can contain its own control plane or service mesh. Only non-sensitive or tokenized data can ever leave the clusters to be aggregated with similar non-sensitive data from other institutional customer environments. The data related to individual consumers or their associated primary users can be logically segmented from each other within cluster databases, or only the consumer admin or any users authorized by the consumer admin can ever see personally identifiable information about the primary user.

A distinction between consumer customers or institutional customers regarding data segmentation can be the sharing of databases or the lack thereof. While consumers within a country can leverage shared databases in which each customer's data is logically separated, institutional customers can have a dedicated Kubernetes fleet with pairs of Kubernetes clusters for each country in which they manage facilities or onboard primary users. This configuration can ensure that all primary user data remains in the country it is generated.

Where institutional customers have multiple facilities, the institutional customers can set policies by facility or groupings of facilities within the institutional admin console. Within a Kubernetes cluster, each facility operated by an institutional customer can have a dedicated namespace. Namespaces can share a control plane or service mesh within a Kubernetes cluster. Still, each microservice can reside within a dedicated pod within a namespace or have its own dedicated databases or sidecar proxies. Whereas Kubernetes defaults to allowing any resource within a cluster to connect with any other resource in that same cluster, the application can have strict security protocols in place to ensure that all network communication within Kubernetes clusters is pre-authorized via comprehensive allowlisting or that unapproved network activity is detected or addressed.

The application platform can be designed to be HIPAA or GDPR-compliant, with plans for SOC2 certification. While a comprehensive list of cybersecurity controls can be provided, certain principles can be applied during MVP development to minimize future rework. These can include implementing a Secure Software Development Life Cycle (SDLC) or toolchain with strict controls, multi-factor authentication, or traceable activity logs. Code quality measures can involve continuous security vulnerability scanning or open-source component approval. Accessor Identity Management can require multi-factor authentication for all platform components, with administrative access logged or recorded securely. Infrastructure Security can encompass programmatic patching, vulnerability scanning, endpoint security, intrusion detection, or device security measures. Web Application Controls can include web application firewall protection or measures against botnet or DDoS attacks. Data Leak Prevention solutions can be implemented with tamper-proof records. Secrets Management can involve data encryption at rest or in transit, with appropriate tools or processes for safeguarding secrets. Credential Management can ensure traceability of credential use, mandate changing default passwords, or align password complexity with industry standards. These principles can aim to establish a robust security foundation for the application platform from its initial development stages.

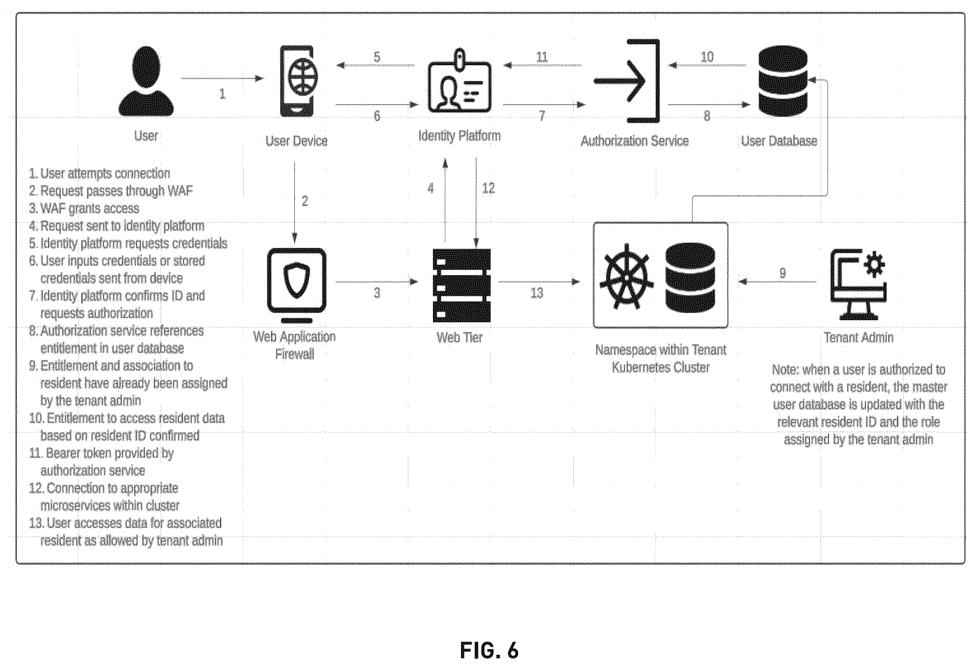

FIG. 6 is a schematic diagram of an example system architecture diagram illustrating the authentication or authorization flow for a user accessing a tenant-specific namespace within a Kubernetes cluster, according to some examples. The process shown in FIG. 6 is an example process that can omit steps, include more steps, or be completed in an order different than as shown herein.

At operation 1, a user can initiate a connection attempt (e.g., via the VestaConnect application). At operation 2, the connection request can pass through a Web Application Firewall (WAF), granting access or transmitting the request to a web tier at operation 3. At operation 4, the web tier can transmit the approved request to an identity platform, which can transmit an identity request for credentials to the user device at operation 5. The user can input credentials, or credentials stored on the user device can be transmitted to the identity platform at operation 6. At operation 7, the identity platform can confirm ID or request authorization from an authorization service.

At operation 8, the authorization service can reference entitlement in a user database. At operation 9, entitlement or association to the resident can be confirmed (e.g., the authorization service can confirm that the tenant admin has already assigned entitlement or association to the resident). The authorization service can receive confirm entitlement to access resident data based on resident ID via the user database. At operation 11, the bearer token can be provided to the identity platform via the authorization service.

Operation 12 can include the web tier providing connection to appropriate microservices within the cluster based on the bearer token. At operation 13, the user can access data for associated residents as the tenant admin allows. In examples, as a user is authorized to connect with a resident, the master User Database can be updated with the relevant resident ID or the role assigned by the Tenant Admin.

This architecture can support a multi-tenant environment where user access is controlled at a granular level, with specific entitlements tied to individual residents. The system can employ a series of checks or balances, from initial WAF screening to identity confirmation or role-based access control, ensuring secure or appropriate data access within the Kubernetes cluster.

The application can include many security solutions, such as those discussed above, to help protect the information of the primary user. Primary users can be registered either by institutional or consumer admins, who then authorize connected user admins or connected users to access services or data related to a primary user. This approval propagates within the applications (e.g., application 104, see FIG. 1) master user database without exposing primary user personal information.

The application can employ numerous cyber security controls to protect the internal IT systems of the smart screen appliance 102 (FIG. 1) and the application 104 (FIG. 1). The smart screen appliance 102 or the application 104 can include comprehensive, real-time monitoring or cyber security operations center services. Moreover, the smart screen appliance 102 or the application 104 can include infrastructure level or code vulnerability scanning to identify or address vulnerabilities. All infrastructure or software components can be patched or monitored for indications of compromise or any other suspicious activity. The smart screen appliance 102 or the application 104 can also include threat intelligence to proactively hunt for indications of malicious intent toward the smart screen appliance 102 or the application 104, customers or users, or active searches for compromised credentials.

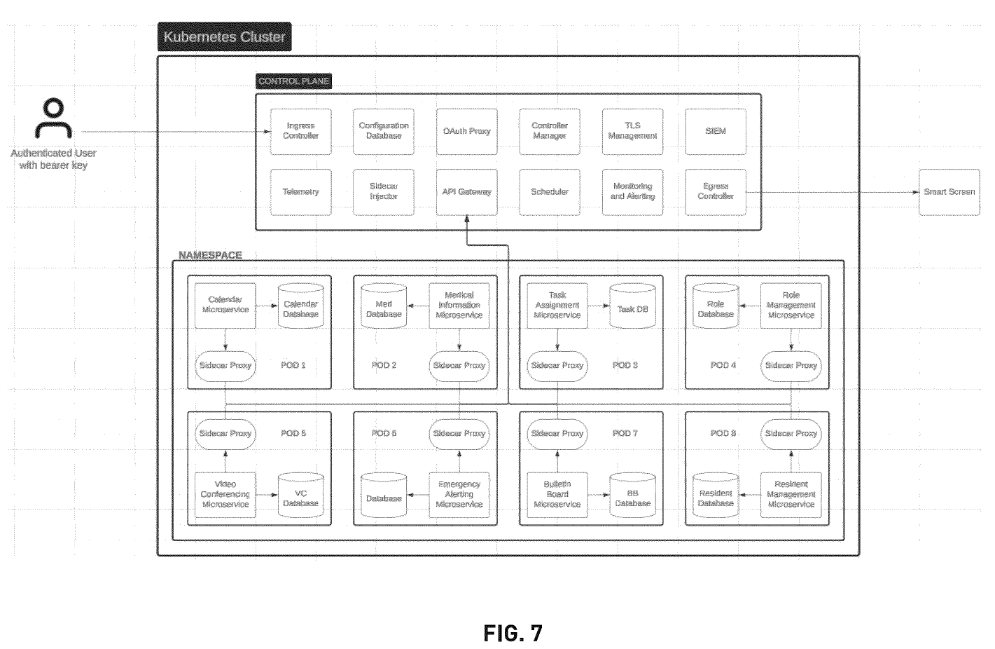

FIG. 7 is a system architecture diagram illustrating the structure or components of a Kubernetes cluster for a multi-tenant application, according to some examples.

As shown in FIG. 7, the Kubernetes Cluster, a complex system with multiple layers, can be interfaced with a smart screen (e.g., the smart screen appliance 102). This smart screen can serve as a gateway to the cluster. The cluster can be divided into two main sections: the control plane or the namespace. The control plane, the brain of the cluster, houses several components that manage the overall cluster operations. These components can include one or more of the following:
A Security Information or Event Management system;
An Egress Controller, which can manage outbound traffic from the cluster;
A TLS Management, which can handle Transport Layer Security protocols;
A monitoring or alert component that can oversee cluster health or transmit notifications;
A Controller Manager, which can regulate the state of the cluster;
A Scheduler that can assign workloads to nodes;
An API Gateway that can manage API requests or routing;
An OAuth Proxy, which can handle authentication or authorization;
An Ingress Controller, which can manage inbound traffic to the cluster; or
A Configuration Database that can store cluster configuration data.

The Namespace section can include multiple pods, each representing a microservice within the application. These pods can include:
POD 1: A Calendar Microservice with a Calendar Database;
POD 2: A Medical Information Microservice with a Med Database;
POD 3: A Task Assignment Microservice with a Task DB;
POD 4: A Role Management Microservice with a Role Database;
POD 5: A Video Conferencing Microservice with a VC Database;
POD 6: A Emergency Alerting Microservice;
POD 7: A Bulletin Board Microservice with a BB Database; or
POD 8: A Resident Management Microservice with a Resident Database.

Each pod, representing a microservice within the application, can contain a sidecar proxy. This sidecar proxy, a component in the system, facilitates communication between microservices or handles cross-cutting concerns such as logging, monitoring, or security. The control plane also includes a sidecar Injector, which automatically injects the sidecar containers into pods as they are created. The system can require an Authenticated User with a bearer key as an entry point for authenticated users accessing the system.

This architecture, designed for a scalable, secure, or modular application within a Kubernetes environment, offers a high degree of flexibility. The separation of concerns between the Control Plane or Namespace allows for the efficient management of cluster-wide operations or tenant-specific services. The use of microservices in individual pods enables independent scaling or updates of different application components, enhancing the system's modularity.

The Calendar Microservice can be a service that can be edited by institutional or consumer admins, as well as connected user admins or institutional staff users, for whom the institutional or consumer admins have granted privileges through their respective consoles. This microservice can offer the ability to create both one-time or recurring events, with recurring events having multiple options for recurrence such as daily, weekly, monthly, or on specific days of the month (1st, 2nd, 3rd, 4th) with either a specific end date or no end date. The Calendar Microservice can allow for individual instances of recurring events to be canceled or rescheduled without affecting other instances in the series. Connected user admins with event creation privileges can be restricted from overwriting or editing events created by institutional or consumer admins. The Calendar Microservice can also allow admins to create optional color coding for event types, which can be configured in their respective admin consoles.

The Task Management Microservice can permit consumer admins, institutional admins, connected user admins, or institutional staff to assign one-time or recurring tasks to primary users. The Primary users can mark tasks as complete by clicking on them, with completed tasks remaining visible until expiration for one-time tasks or until reset for recurring tasks. The task management microservice can offer recurrence options similar to the calendar service. The admins or institutional staff can mark tasks as complete or reset them on behalf of primary users. The institutional admins can assign tasks to individual primary users or groups. One-time tasks can include configurable default expirations. If they have proper permissions, Connected User Admins can create or edit tasks but cannot modify those created by institutional admins or staff.

The Medical Information Microservice can provide a free text field for each primary user where consumer or institutional admins can enter medication history, allergies, or medication schedules. In examples, only the admins can add or edit information, but institutional admins can grant viewing or editing rights to connected user admins or institutional staff. Activity related to adding or editing this information can be logged or available to institutional admins, including time, details of changes, or who made them.

The Video or Audio Calling Microservice can enable primary residents to initiate calls with contacts from their home screen. Admins can configure calling options, including hours or call types. Connected User Admins or Users can appear on the Contact List by default, or admins can have the ability to manage contacts.

The Noticeboard Microservice can allow admins to post informational items for primary users, with potential options for one-time or recurring posts.

The Voting Microservice can enable admins to create votes for primary users, with configurable options for recurrence, expiration, or visibility.

The Request Help Microservice can provide a constantly visible help button for primary users, with configurable actions when pressed.

The Smart Screen Management Microservice can handle onboarding, assigning, or managing smart screen appliances.

The Primary User Management Microservice can allow admins to create or manage primary user accounts.

The Role Management Microservice can enable admins to assign roles to connected users.

The Institutional Staff Management Microservice can create or manage institutional staff accounts.

The User Registration or Management Microservices can handle account creation, security controls, or ongoing account management for all users.

Finally, the Institution Onboarding or Management Microservices can facilitate institutional customer onboarding or ongoing management.

These microservices are just examples of microservices that can be used in the patient care system 100 (FIG. 1) within the application 104 (FIG. 1) on the smart screen appliance 102 (FIG. 1). The inventors of the present disclosure contemplate many additional microservices that can be used, or this list is not intended to be limited to just those disclosed.

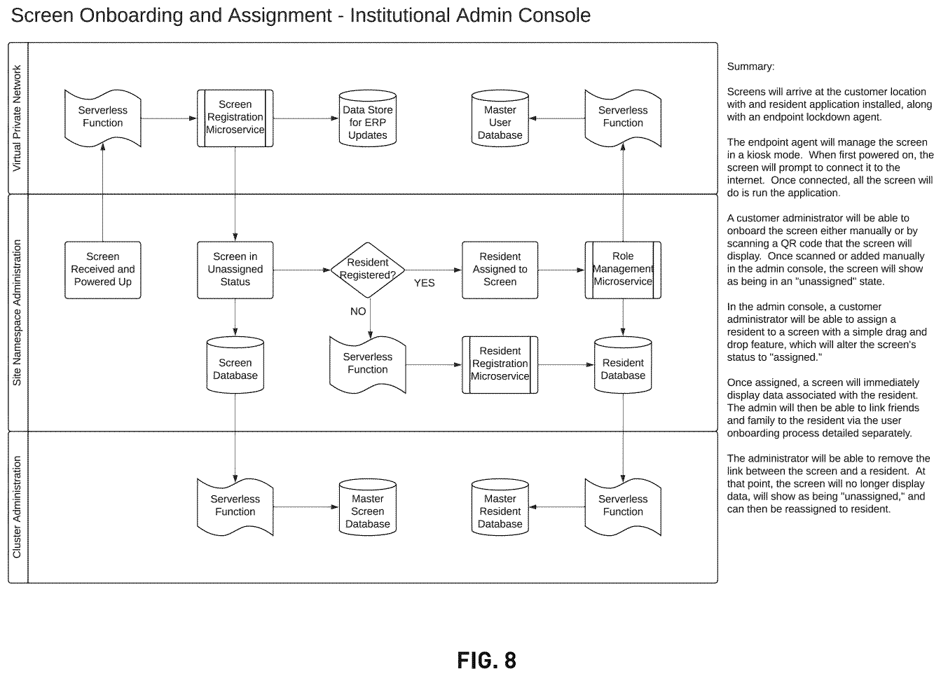

FIG. 8 is a schematic diagram illustrating an example of the screen onboarding or assignment process for an institutional admin console, according to some examples of the patient care system 100 (FIG. 1). The process shown in FIG. 8 is an example process that can omit steps, include more steps, or be completed in an order different than as shown herein.

The patient care system 100 can include three sections: Virtual Private Network, Site Namespace Administration, or Cluster Administration. Each section can represent a different level of the system architecture or can contain various components that interact to facilitate the screen onboarding or assignment process.

The Virtual Private Network section can include the Serverless Function. This function triggers when a Screen is Received or Powered Up, initiating the screen registration process. It also interacts with a Data Store for ERP Updates or communicates with a Master User Database through another Serverless Function, playing a role in the screen onboarding or assignment process.

After registration, the screen can enter an Unassigned Status, which can be recorded in a Screen Database. A decision point can then check if a Resident is Registered. If not, a Serverless Function can trigger the Resident Registration Microservice, which can interact with a Resident Database. The process can move to Resident Assigned to Screen if a resident is registered or after registration. This assignment can trigger the Role Management Microservice to update the Resident Database.

The patient care system 100 can also include Serverless Functions that can interact with a Master Screen Database or a Master Resident Database. These databases can likely contain aggregated data from multiple sites or institutions within the cluster.

The patient care system 100 can be designed to handle a one-to-many relationship between screens or residents. This means that while a screen can be assigned to only one resident at a time, a resident's information can potentially be associated with multiple screens over time. This design allows for flexibility in managing resident assignments or ensures that all relevant information is easily accessible.

The patient care system 100 can also include support for the operations of screen onboarding or resident assignment in a care home management system. It can allow for the registration of new screens, the assignment of residents to screens, or the management of roles or permissions associated with these assignments.

The patient care system 100 can also integrate other system components by showing how the screen onboarding process interacts with various microservices, databases, or serverless functions across different levels of the system architecture. It can demonstrate the flow of data or processes from the initial screen setup to the final assignment or role management.

Alternative configurations can exist for different types of institutions or consumer-oriented versions of the system. These can involve different microservices, additional decision points, or varied database structures to accommodate different requirements or user types.

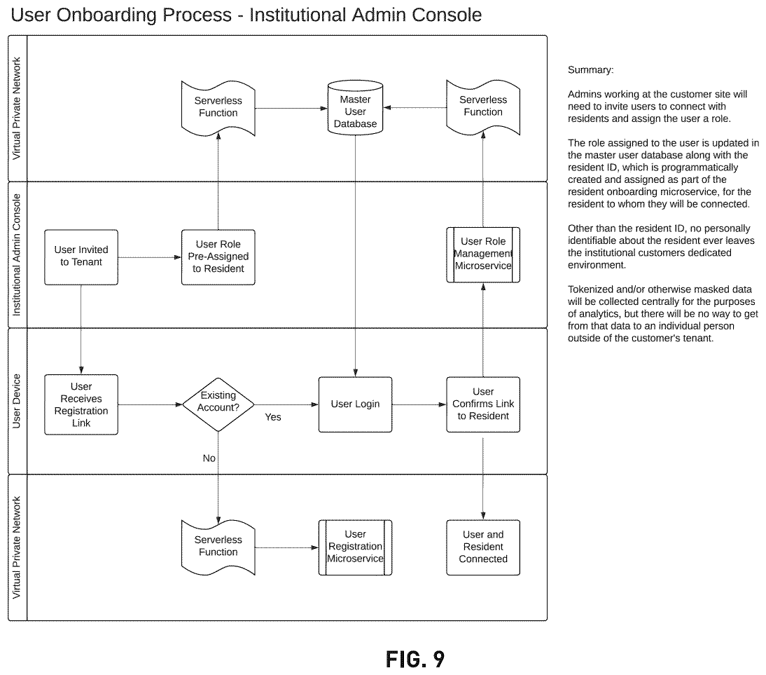

FIG. 9 is a process flow diagram illustrating the user onboarding process for an institutional admin console, according to some examples of the patient care system 100. The process shown in FIG. 9 is an example process that can omit steps, include more steps, or be completed in an order different than as shown herein.

The onboarding process can include four main sections: Virtual Private Network, Institutional Admin Console, User Device, or a lower Virtual Private Network section. Each section can represent a different layer or component of the system involved in the user onboarding process.

The Virtual Private Network can include two Serverless Functions that can interact with a Master User Database. These functions can handle data processing or updates related to user information.

The Institutional Admin Console can include a “User Invited to Tenant,” followed by “User Role Pre-Assigned to Resident.” Thus, the institutional admin can initiate the process by inviting a user or pre-assigning their role to a resident.

The User Device can include the user's interaction with the system. The User Device can begin with “User Receives Registration Link,” followed by an “Existing Account?” decision point. The flow can proceed to “User Login” if the user has an existing account. If not, the process can continue to the lower Virtual Private Network section. After login or new account creation, the user can confirm their link to a resident through “User Confirms Link to Resident.”

The Virtual Private Network can include a serverless function for users without existing accounts that triggers the User Registration Microservice to create new user accounts.

The system can include several one-to-many relationships. For example, one User Role Management Microservice can handle multiple user role assignments, or one Master User Database can store information for multiple users.

The user onboarding process can integrate with other system components by interacting with the Master User Database or utilizing microservices or serverless functions. The user onboarding process can support user management, role assignment, or establishing connections between users or residents in a care home management system.

The role assigned to the user can be updated in the master user database along with the resident ID, which can be programmatically created or assigned as part of the resident onboarding microservice for the resident to whom they will be connected.

Besides the resident ID, no personally identifiable information about the resident can leave the institutional customer's dedicated environment. Tokenized or otherwise masked data can be collected centrally for analytics, but that data cannot be obtained from a person outside the customer's tenant.

Alternative configurations of this process can exist for different types of users or institutions. For instance, the role assignment process can vary or include additional steps for specific types of users or facilities.

FIG. 10-FIG. 30 are examples of graphical user interfaces that may be used with or to implement the systems or techniques described herein.

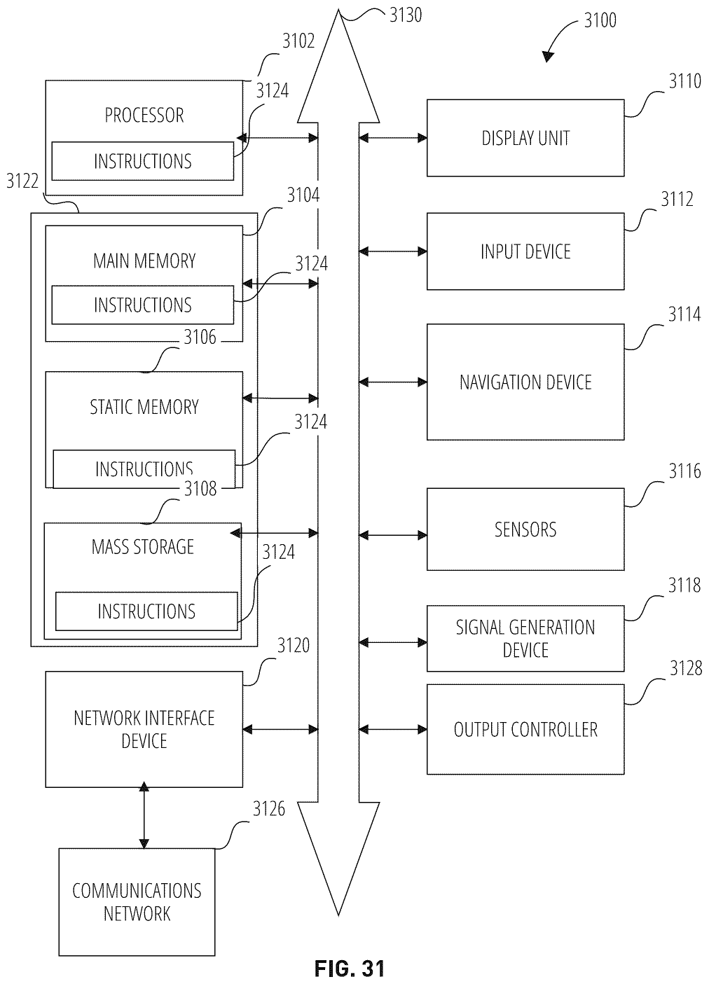

FIG. 31 illustrates a block diagram of an example machine 3100 upon which any one or more of the techniques (e.g., methodologies) discussed herein can perform. Examples, as described herein, can include, or can operate by, logic or a number of components, or mechanisms in the machine 3100. Circuitry (e.g., processing circuitry) is a collection of circuits implemented in tangible entities of the machine 3100 that include hardware (e.g., simple circuits, gates, logic, etc.). Circuitry membership can be flexible over time. Circuitries include members that can, alone or in combination, perform specified operations when operating. In an example, hardware of the circuitry can be immutably designed to carry out a specific operation (e.g., hardwired). In an example, the hardware of the circuitry can include variably connected physical components (e.g., execution units, transistors, simple circuits, etc.), including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components can be used in more than one member of more than one circuitry. For example, under operation, execution units can be used in a first circuit of a first circuitry at one point in time or reused by a second circuit in the first circuitry, or by a third circuit in a second circuitry at a different time. Additional examples of these components with respect to the machine 3100 follow.

In alternative examples, the machine 3100 can operate as a standalone device or can be connected (e.g., networked) to other machines. In a networked deployment, the machine 3100 can operate in the capacity of a server machine, a client machine, or both in server-client network environments. In an example, the machine 3100 can act as a peer machine in peer-to-peer (P2P) (or other distributed) network environment. The machine 3100 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 3100 can include a hardware processor 3102 (e.g., a central processing unit (CPU), a graphics processing unit (GPU), a hardware processor core, or any combination thereof), a main memory 3104, a static memory (e.g., memory or storage for firmware, microcode, a basic-input-output (BIOS), or mass storage 3108 (e.g., hard drives, tape drives, flash storage, or other block devices) some or all of which can communicate with each other via an interlink 3130 (e.g., bus). The machine 3100 can further include a display unit 3110, an alphanumeric input device 3112 (e.g., a keyboard), or a user interface (UI) navigation device 3114 (e.g., a mouse). In examples, the display unit 3110, input device 3112orUI navigation device 3114 can be a touch screen display. The machine 3100 can additionally include a signal generation device 3118 (e.g., a speaker), a network interface device 3120, or one or more sensors 3116, such as a global positioning system (GPS) sensor, compass, accelerometer, or other sensor. The machine 3100 can include an output controller 3128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 3102, the main memory 3104, the static memory 3106, or the mass storage 3108 can be, or include, a machine-readable medium 3122 on which is stored one or more sets of data structures or instructions 3124 (e.g., software) embodying or utilized by any one or more of the techniques or functions described herein. The instructions 3124 can also reside, completely or at least partially, within any of registers of the processor 3102, the main memory 3104, the static memory 3106, or the mass storage 3108 during execution thereof by the machine 3100. In an example, one or any combination of the hardware processor 3102, the main memory 3104, the static memory 3106, or the mass storage 3108 can constitute the machine-readable media 3122. While the machine-readable medium 3122 is illustrated as a single medium, the term “machine-readable medium” can include a single medium or multiple media (e.g., a centralized or distributed database, and/or associated caches or servers) configured to store the one or more instructions 3124.

The term “machine-readable medium” can include any medium that is capable of storing, encoding, or carrying instructions for execution by the machine 3100 or that cause the machine 3100 to perform any one or more of the techniques of the present disclosure, or that is capable of storing, encoding or carrying data structures used by or associated with such instructions. Non-limiting machine-readable medium examples can include solid-state memories, optical media, magnetic media, or signals (e.g., radio frequency signals, other photon-based signals, sound signals, etc.). In an example, a non-transitory machine-readable medium comprises a machine-readable medium with a plurality of particles having invariant (e.g., rest) mass, or thus are compositions of matter. Accordingly, non-transitory machine-readable media are machine-readable media that do not include transitory propagating signals. Specific examples of non-transitory machine-readable media can include: non-volatile memory, such as semiconductor memory devices (e.g., Electrically Programmable Read-Only Memory (EPROM), Electrically Erasable Programmable Read-Only Memory (EEPROM)) or flash memory devices; magnetic disks, such as internal hard disks or removable disks; magneto-optical disks; orCD-ROMorDVD-ROM disks.

In an example, information stored or otherwise provided on the machine-readable medium 3122 can be representative of the instructions 3124, such as instructions 3124 themselves or a format from which the instructions 3124 can be derived. This format from which the instructions 3124 can be derived can include source code, encoded instructions (e.g., in compressed or encrypted form), packaged instructions (e.g., split into multiple packages), or the like. The information representative of the instructions 3124 in the machine-readable medium 3122 can be processed by processing circuitry into the instructions to implement any of the operations discussed herein. For example, deriving the instructions 3124 from the information (e.g., processing by the processing circuitry) can include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 3124.

In an example, the derivation of the instructions 3124 can include assembly, compilation, or interpretation of the information (e.g., by the processing circuitry) to create the instructions 3124 from some intermediate or preprocessed format provided by the machine-readable medium 3122. The information, when provided in multiple parts, can be combined, unpacked, or modified to create the instructions 3124. For example, the information can be in multiple compressed source code packages (object code, or binary executable code, or the like) on one or several remote servers. The source code packages can be encrypted when in transit over a network or decrypted, uncompressed, assembled (e.g., linked) if necessary, or compiled or interpreted (e.g., into a library, stand-alone executable etc.) at a local machine, or executed by the local machine.

The instructions 3124 can be further transmitted or received over a communications network 3126 using a transmission medium via the network interface device 3120 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, or wireless data networks (e.g., Institute of Electrical or Electronics Engineers (IEEE) 802.11 family of standards known as Wi-Fi®, IEEE 802.15.4 family of standards, peer-to-peer (P2P) networks, among others. In an example, the network interface device 3120 can include one or more physical jacks (e.g., Ethernet, coaxial, or phone jacks) or one or more antennas to connect to the communications network 3126. In an example, the network interface device 3120 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 3100, or includes digital or analog communications signals or other intangible medium to facilitate communication of such software. A transmission medium is a machine-readable medium.

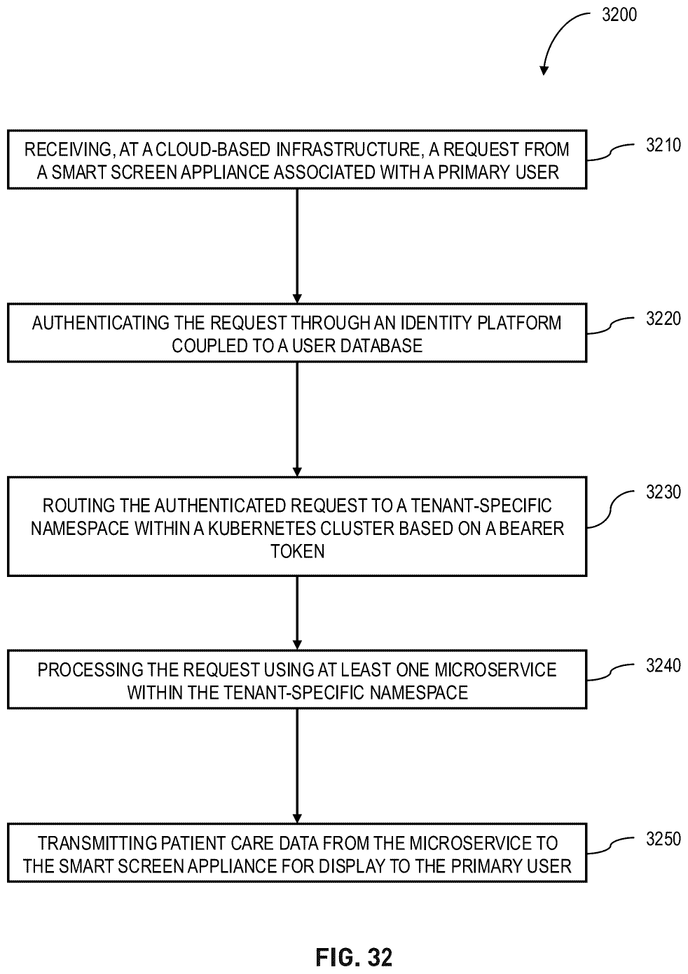

FIG. 32 illustrates a flowchart of an example method 3200 for a patient care system. The method 3200 can be implemented within the patient care system 100 described herein to facilitate secure, multi-tenant patient care management. In examples, the method 3200 can include any one or more of the operations 3210-3250.

At operation 3210, method 3200 can include receiving a request from a smart screen appliance associated with a primary user. The request can be received at a cloud-based infrastructure through a web application firewall (WAF) that mediates all access to the application and protects against distributed denial of service or bot attacks. The smart screen appliance can be operating in kiosk mode, locked down to only run the patient care application, with an endpoint lockdown agent that forces the device into this restricted mode. The request can include user interaction data from the touchscreen display, such as calendar access requests, task completion confirmations, help requests, or video call initiation commands.

At operation 3220, the method 3200 can include authenticating the request through an identity platform coupled to a user database. The authentication process can require multi-factor authentication, as no access to any component of the application platform is permitted without MFA. The identity platform can confirm user credentials and reference a master user database that contains only ID numbers and non-personally identifiable or tokenized data about primary users to ensure privacy protection. The authentication can include checking for anomalous behavior such as unexpected geography or unusual access times, which can force additional MFA or revocation of relevant tokens.

At operation 3230, the method 3200 can include routing the authenticated request to a tenant-specific namespace within a Kubernetes cluster based on a bearer token. The authorization service can reference entitlement in the user database and confirm association to the resident based on resident ID. For institutional customers, each facility can have a dedicated namespace within the Kubernetes cluster, while consumer customers can leverage shared databases with logically separated data. The bearer token can include role-based access control information that determines which microservices and data the user can access within the namespace.

At operation 3240, the method 3200 can include processing the request using at least one microservice within the tenant-specific namespace. The microservices can include a calendar microservice for managing scheduling information, a medical information microservice for storing patient medical data, a task assignment microservice for managing patient tasks, a video conferencing microservice for audio/video communication, or other specialized microservices. Each microservice can reside within a dedicated pod with its own dedicated database and sidecar proxy for secure inter-service communication. The microservice can process the request according to the user's permissions and the specific functionality requested.

At operation 3250, the method 3200 can include transmitting patient care data from the microservice to the smart screen appliance for display to the primary user. The transmitted data can include calendar information with color-coded events, task lists with completion status, medical reminders, weather information, current date and time, or communication interfaces for video/audio calls. The smart screen appliance can render a user interface including the data and output it on the touchscreen display in a simplified, user-friendly format with large, clear icons and simple navigation suitable for users with cognitive challenges. If network connectivity is interrupted, the smart screen appliance can utilize its local cache to maintain basic functionality until connectivity is restored.

The following non-limiting examples detail certain aspects of the present subject matter to solve the challenges or provide the benefits discussed herein, among others.

Example 1 is a patient care system comprising: a smart screen appliance including a touchscreen display and processing circuitry configured to execute a patient care application; a cloud-based infrastructure including a plurality of microservices deployed in a multi-tenant architecture, the plurality of microservices configured to manage patient care data, the cloud-based infrastructure including: a web tier, the smart screen appliance configured to communicate with the cloud-based infrastructure via the web tier; and a plurality of customer-specific clusters, each customer-specific cluster of the plurality of customer-specific clusters including a control plane and a plurality of namespaces, each namespace of the plurality of namespaces including at least one microservice of the plurality of microservices and an associated database; and an administrative console in communication with the cloud-based infrastructure through the web tier to access data from the plurality of microservices.

In Example 2, the subject matter of Example 1 optionally includes wherein the smart screen appliance includes an endpoint lockdown agent configured to restrict operation of the smart screen appliance to executing only the patient care application.

In Example 3, the subject matter of any one or more of Examples 1-2 optionally include wherein the smart screen appliance includes a local cache configured to maintain functionality during network connectivity interruptions with the cloud-based infrastructure.

In Example 4, the subject matter of any one or more of Examples 1-3 optionally include wherein the plurality of microservices includes a calendar microservice configured to manage scheduling information for patients.

In Example 5, the subject matter of Example 4 optionally includes wherein the calendar microservice is configured to create events for each patient, and wherein each calendar event for each patient can include color coding for different event types.

In Example 6, the subject matter of any one or more of Examples 1-5 optionally include wherein the plurality of microservices includes a medical information microservice configured to store and manage patient medical data.

In Example 7, the subject matter of Example 6 optionally includes wherein the patient medical data includes at least one of prescription drug schedules, doctor appointments, care schedules, reminders related to health screenings, and recorded medical readings.

In Example 8, the subject matter of Example 7 optionally includes wherein the medical information microservice is configured to log all access and modifications to the patient medical data as logged information.

In Example 9, the subject matter of Example 8 optionally includes wherein the logged information includes timestamps, details of changes, and user identification.

In Example 10, the subject matter of any one or more of Examples 1-9 optionally include wherein the plurality of microservices includes a task assignment microservice configured to manage tasks assigned to a primary user.

In Example 11, the subject matter of Example 10 optionally includes wherein the task assignment microservice is configured to create recurring tasks with configuration expiration settings, and wherein a completed task remains visible until expiration for one-time tasks or until reset for recurring tasks.

In Example 12, the subject matter of any one or more of Examples 1-11 optionally include wherein the touchscreen display is configured to display a barcode, wherein scanning the barcode provides emergency access to medical information related to a primary user.

In Example 13, the subject matter of Example 12 optionally includes wherein the medical information includes at least one of medication history, allergies, medication schedules, and therapy schedules.

In Example 14, the subject matter of any one or more of Examples 1-13 optionally include wherein the plurality of microservices includes a video conferencing microservice configured to enable audio and video communication, and wherein the video audio microservice is configured to provide unidirectional and bidirectional call initiation options.

In Example 15, the subject matter of Example 14 optionally includes wherein the video conferencing microservice is configured to generate an alert if an activity is underway before initiating a call, or before the activity starts during the call.

Example 16 is a method for providing a smart screen appliance for patient care, the method comprising: receiving, at a cloud-based infrastructure, a request from a smart screen appliance associated with a primary user; authenticating the request through an identity platform coupled to a user database; routing the authenticated request to a tenant-specific namespace within a Kubernetes cluster based on a bearer token; processing the request using at least one microservice within the tenant-specific namespace; and transmitting patient care data from the microservice to the smart screen appliance for display to the primary user.

In Example 17, the subject matter of Example 16 optionally includes wherein the tenant-specific namespace includes a plurality of microservices each deployed in separate pods with dedicated databases and sidecar proxies for secure inter-service communication.

In Example 18, the subject matter of any one or more of Examples 16-17 optionally include retrieving documents associated with the primary user from a document management microservice; categorizing the documents based on assigned tags corresponding to scheduled activities; and automatically delivering relevant documents to the smart screen appliance based on an activity schedule of the primary user.

In Example 19, the subject matter of any one or more of Examples 16-18 optionally include monitoring user activity on the smart screen appliance; detecting a period of user inactivity exceeding a predetermined threshold; and automatically returning the smart screen appliance to a default display view showing basic patient information.

In Example 20, the subject matter of any one or more of Examples 16-19 optionally include receiving an emergency code input at the smart screen appliance; activating an emergency mode in response to the emergency code input; capturing a photograph using the smart screen appliance during emergency mode activation; retrieving comprehensive medical information for the primary user from a medical information microservice; and transmitting emergency notifications to predetermined contacts while displaying the medical information.

Example 21 can optionally include a method, system, patient care device, mobile smart device, or other, that includes any element of any one or more of Examples 1-20.

The above-detailed description includes references to the accompanying drawings, which form a part of the detailed description. The drawings show, by way of illustration, specific examples that can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, or patent documents referred to in this document are incorporated by reference herein in their entirety, as though individually incorporated by reference. In the event of inconsistent usages between this document or those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” or “A or B,” unless otherwise indicated. In the appended claims, the terms “including” or “in which” are used as the plain-English equivalents of the respective terms “comprising” or “wherein.” Also, in the following claims, the terms “including” or “comprising” are open-ended, that is, a system, device, article, or process that includes elements in addition to those listed after such a term in a claim are still deemed to fall within the scope of that claim. Moreover, in the following claims, the terms “first,” “second,” or “third,” etc., are used merely as labels or are not intended to impose numerical requirements on their objects.

The term “about,” as used herein, means approximately, in the region of, roughly, or around. When the term “about” is used in conjunction with a numerical range, it modifies that range by extending the boundaries above or below the numerical values set forth. In general, the term “about” is used herein to modify a numerical value above or below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers or fractions subsumed within that range (e.g., 1 to 5 includes 1, 1.5, 2, 2.75, 3, 3.90, 4, 4.24, or 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, I-4, or 2-4). It is also to be understood that all numbers or fractions thereof are presumed to be modified by the term “about.”

The above description is intended to be illustrative, or not restrictive. For example, the above-described examples (or one or more aspects thereof) can be used in combination with each other. Other examples can be used, such as one of ordinary skill in the art upon reviewing the above description. The Abstract is to allow the reader to quickly ascertain the nature of the technical disclosure or is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

What is claimed is:

1. A patient care system comprising:
a smart screen appliance including a touchscreen display and processing circuitry configured to execute a patient care application;
a cloud-based infrastructure including a plurality of microservices deployed in a multi-tenant architecture, the plurality of microservices configured to manage patient care data, the cloud-based infrastructure including:
a web tier, the smart screen appliance configured to communicate with the cloud-based infrastructure via the web tier; and
a plurality of customer-specific clusters, each customer-specific cluster of the plurality of customer-specific clusters including a control plane and a plurality of namespaces, each namespace of the plurality of namespaces including at least one microservice of the plurality of microservices and an associated database; and
an administrative console in communication with the cloud-based infrastructure through the web tier to access data from the plurality of microservices.

2. The patient care system of claim 1, wherein the smart screen appliance includes an endpoint lockdown agent configured to restrict operation of the smart screen appliance to executing only the patient care application.

3. The patient care system of claim 1, wherein the smart screen appliance includes a local cache configured to maintain functionality during network connectivity interruptions with the cloud-based infrastructure.

4. The patient care system of claim 1, wherein the plurality of microservices includes a calendar microservice configured to manage scheduling information for patients.

5. The patient care system of claim 4, wherein the calendar microservice is configured to create events for each patient, and wherein each calendar event for each patient can include color coding for different event types.

6. The patient care system of claim 1, wherein the plurality of microservices includes a medical information microservice configured to store and manage patient medical data.

7. The patient care system of claim 6, wherein the patient medical data includes at least one of prescription drug schedules, doctor appointments, care schedules, reminders related to health screenings, and recorded medical readings.

8. The patient care system of claim 7, wherein the medical information microservice is configured to log all access and modifications to the patient medical data as logged information.

9. The patient care system of claim 8, wherein the logged information includes timestamps, details of changes, and user identification.

10. The patient care system of claim 1, wherein the plurality of microservices includes a task assignment microservice configured to manage tasks assigned to a primary user.

11. The patient care system of claim 10, wherein the task assignment microservice is configured to create recurring tasks with configuration expiration settings, and wherein a completed task remains visible until expiration for one-time tasks or until reset for recurring tasks.

12. The patient care system of claim 1, wherein the touchscreen display is configured to display a barcode, wherein scanning the barcode provides emergency access to medical information related to a primary user.

13. The patient care system of claim 12, wherein the medical information includes at least one of medication history, allergies, medication schedules, and therapy schedules.

14. The patient care system of claim 1, wherein the plurality of microservices includes a video conferencing microservice configured to enable audio and video communication, and wherein the video audio microservice is configured to provide unidirectional and bidirectional call initiation options.

15. The patient care system of claim 14, wherein the video conferencing microservice is configured to generate an alert if an activity is underway before initiating a call, or before the activity starts during the call.

16. A method for providing a smart screen appliance for patient care, the method comprising:
receiving, at a cloud-based infrastructure, a request from a smart screen appliance associated with a primary user;
authenticating the request through an identity platform coupled to a user database;
routing the authenticated request to a tenant-specific namespace within a Kubernetes cluster based on a bearer token;
processing the request using at least one microservice within the tenant-specific namespace; and
transmitting patient care data from the microservice to the smart screen appliance for display to the primary user.

17. The method of claim 16, wherein the tenant-specific namespace includes a plurality of microservices each deployed in separate pods with dedicated databases and sidecar proxies for secure inter-service communication.

18. The method of claim 16, comprising:
retrieving documents associated with the primary user from a document management microservice;
categorizing the documents based on assigned tags corresponding to scheduled activities; and
automatically delivering relevant documents to the smart screen appliance based on an activity schedule of the primary user.

19. The method of claim 16, comprising:
monitoring user activity on the smart screen appliance;
detecting a period of user inactivity exceeding a predetermined threshold; and
automatically returning the smart screen appliance to a default display view showing basic patient information.

20. The method of claim 16, comprising:
receiving an emergency code input at the smart screen appliance;
activating an emergency mode in response to the emergency code input;
capturing a photograph using the smart screen appliance during emergency mode activation;
retrieving comprehensive medical information for the primary user from a medical information microservice; and
transmitting emergency notifications to predetermined contacts while displaying the medical information.
